SEQUENCE LISTING (13 sequences)

SEQ ID NO: 1 - DNA, 25 bp, Artificial
  primer for PCR
gaattcccgtgcgcccgttcgacgc

SEQ ID NO: 2 - DNA, 25 bp, Artificial
  Primer for PCR
ggatccgtgttgcgggcgatgcgcg

SEQ ID NO: 3 - DNA, 25 bp, Artificial
  Primer for PCR
aagcttagcgtgtccaggcccgctc

SEQ ID NO: 4 - DNA, 26 bp, Artificial
  Primer for PCR
ggatccgacgcaggcgcgcatcgact

SEQ ID NO: 5 - DNA, 29 bp, Artificial
  Primer for PCR
gtcgaattcagcaccgcgcgaaagcccag

SEQ ID NO: 6 - DNA, 29 bp, Artificial
  Primer for PCR
gtcaagctttaagctggagcagctgctac

SEQ ID NO: 7 - protein, 328 aa, Saccharopolyspora erythraea
MPREIDVQDYAKGVLGGSRAKLAQAITLVESTRAEHRAKAQELLVELLPHSGGAHRVGITGVPGVGKSTFIESLGTMLTAQGHRVAVLAVDPSSTRSGGSILGDKTRMPKFASDSGAFVRPSPSAGTLGGVARATRETIVLMEAAGFDVVLVETVGVGQSEVAVAGMVDCFLLLTLARTGDQLQGIKKGVLELADLVAVNKADGPHEGEARKAARELRGALRLLTPVSTSWRPPVVTCSGLTGAGLDTLWEQVEQHRATLTETGELAEKRSRQQVDWTWALVRDQLMSDLTRHPAVRRIVDEVESDVRAGELTAGIAAERLLDAFRER

SEQ ID NO: 8 - DNA, 987 bp, Saccharopolyspora erythraea
atgccgcgcgagatcgacgtccaggactacgccaagggcgtgctcggcggctcgcgcgccaagctggcgcaggcgatcacgctggtggagtcgaccagggccgagcaccgcgcgaaagcccaggaactgctcgtcgagctgctgccgcacagcggtggggcgcaccgggtgggcatcaccggcgtgcccggcgtcggcaagtcgacgttcatcgagtcgctgggcacgatgctgaccgcgcaggggcaccgggtcgcggtgctggcggtcgacccgtcgtccacgcgcagcggcggcagcatcttgggcgacaagacgcggatgcccaagttcgcctccgactccggcgcgttcgtgcggccctccccctcggcgggcacgctcggcggcgtcgcgcgcgcgacccgcgagacgatcgtgctgatggaggcggccggattcgacgtcgtgctcgtggaaacggtgggcgtcggccagtccgaggtcgccgtggcgggaatggtcgactgcttcctgctgctgacgctggcccgcaccggcgaccagttgcagggcatcaagaagggtgtgttggagctggccgaccttgtcgcggtgaacaaggccgacggaccgcacgagggcgaggcgcgcaaggcggcccgcgagctgcgcggcgcgctgcggctgctgaccccggtcagcacgtcgtggagacccccggtggtgacctgcagcggcctgaccggagcgggcctggacacgctctgggagcaggtcgagcagcaccgcgccaccctcaccgagaccggcgagctggccgagaagcgcagccgccagcaggtcgactggacctgggcgctggtgcgcgaccagctcatgtccgacctgacccggcacccggcggtgcgccgcatcgtcgacgaggtcgaatccgacgtgcgggccggggaactgaccgcgggcatcgccgccgagcggctgctcgacgccttccgggagcgctga

SEQ ID NO: 9 - protein, 618 aa, Saccharopolyspora erythraea
MAHSTTSDGPELPLAAEFPEPARQQWRQQVEKVLRRSGLLPEGRPAPEPVEDVLASATYDGITVHPLYTEGPASSGVPGLAPYVRGSRAQGCVSEGWDVRQHHAHPDASETNREILADLYNGTTSLWLELGPTGLPVDSLADALEGVHLDMIGVVLDAGDEAARAASALLELAREQGVRPSALRANLGADPLSTWARTGQERDLGLAAEVAAHCASHPGLRAITVDGLPYHEAGGSDAEELGCSIAAGVTYLRVLAGELGAEAASGLLEFRYAATADQFLTIAKLRAARRLWERVTREIGVAERAQLQHAVTSSAMLTRRDPWVNMLRTTIATFAAGVGGARSVTVRPFDAAIGLPDPFSRRIARNTQSLLLEESHLAQVIDPAGGSWYVETLTDELAHKAWEWFRRIEAEGGLPAALRSGLVADRLAETWQRRRDAVAHRTDPITGVTEFPNLEEPALRRDPAPEPLSGGLPRHRYAEDFERLRDASDAHLAETGARPKVFLATLGSLAEHNARASFARNLFGAGGLETPDAGPTESTEDVVKAFAGSGTPVACLCSGDRIYGEHAEETARALREAGADQVLLAGSLEVPGVDGRVFGGCNALEVLQDVHRRLGVQQ

SEQ ID NO: 10 - protein, 764 aa, Saccharopolyspora erythraea
MTAHEHEPIPSFAGVELGEPAPAPAGRWNDALLAETGKEADALVWEAPEGIGVKPLYTEADTRGLDFLRTYPGIAPFLRGPYPTMYVNQPWTVRQYAGFSTAEQSNAFYRRNLAAGQKGLSVAFDLATHRGYDSDHPRVGGDVGMAGVAIDSIYDMRRLFDGIPLDRMSVSMTMNGAVLPVMALYIVAAEEQGVAPEKLAGTIQNDILKEFMVRNTYIYPPQPSMRIISDIFAYASRRMPKFNSISISGYHIQEAGATADLELAYTLADGVEYLRAGRQAGLDIDSFAPRLSFFWGIGMNFAMEVAKLRAARLLWAKLVKRFEPSDPKSLSLRTHSQTSGWSLTAQDVYNNVVRTCVEAMAATQGHTQSLHTNALDEALALPTDFSARIARNTQLVLQQESGTTRVIDPWGGSHYIERLTQDLAERAWAHITEVEDAGGMAQAIDAGIPKMRIEEAAARTQARIDSGRQPLIGVNKYRYDGDEQIEVLKVDNAGVRAQQLDKLRRLREERDSEACETALRRLTGAAEAALEDNRPDDLAHNLLTLAVDAARHKATVGEISDALEKVFGRHSGQIRTISGVYREESGTSESLERARRKVEEFDEAEGRRPRILVAKMGQDGHDRGQKVIATAFADIGFDVDVGPLFQTPAEVARQAVESDVHVVGVSSLAAGHLTLVPALRDELAGLGRSDIMIVVGGVIPPADFDALRQGGASAIFPPGTVIADAALGLLDQLRAVLDHPAPGEPAGESDGARGGSPGETSSAG

SEQ ID NO: 11 - protein, 119 aa, Saccharopolyspora erythraea
MLAVTVDPNSAVAPFEQVRTQIAQQINDRVLPVGTKLPTVRRLAADLGIAANTAAKAYRELEQAGLIETRGRAGTFVGSAGERSNERAAEAAAEYARTVAALGIPREEALAIVRAALRA

SEQ ID NO: 12 - DNA, 1147 bp, Saccharopolyspora erythraea
ccatcgtgccgcccatcgtgcacggctgccgcgaaccggcgcggagcagccgcgataccgcgcggcgaagccgaatccgacatgttcgcactccgcgcgcgtgcgcggcaccgccgtgcaacggtgaattcaccagccgagcggctgtgtcgcgcggaccggcggcggccatagcctggccgcgggcgcacgatccgctgcgcgccagggagaaccgcgcgctacggaggtcgccatgtccggccacggccaatcggacggcaccgcgtcgagccggccgtgcgaggactcccgcgccgaggtggaggccctgctgcggtccggtcccttccacgaggcgctgcgcgcggccatcgcgcacagcggactcaccctggaggccctgcgcggtgaactggccgcgcgcggcatccggctcagcctggcgaccctgagctactggcagcacgggcgaagccgccccgagcggaccggctcgatgctggcgctgcgcgcgatcgagaacatcctgcggctgcccgcgcattcgctgcgcgcgctgctgggtccgccgcgcccgcgcggccggtggctcaaccacgagcccggccgcggcatcgacgaccccgccgggcagctcgcggaggtgatcgggccggtgctggggccgtccgaccgcgacctgcgcgtcttctcccaggaggacatcgcctccgtcggcccggaccgggcgatccacctggtgcgtacccgcacggtgctgcgcgcgctggccgacgggcccgaccgccacctcgccgtctaccgcggcgaacccggcaccgactcgggcgcgctggtcccggtcgccaccgagaactgccggctcggccggaccagcaggcacccggccgccccgatcgtggtcgccgagctgttgttcgaccgcaggatgcgcgccggggagacccacctgctggagtacgagttccgcgtcgagcgcccggtgcgcagcgtcgaccaccgccgcacgttccggtacccggcgggcagctacgtcgcgtcggtgcggttctcggagtcggcggtcccggtgcggtgcaggcggctgcgccaaggcgcaccggctgccgggcgcgggaccgacgagctgacactggtgggtggtcgttcggtgcacctcgcggtg

SEQ ID NO: 13 - DNA, 1449 bp, Saccharopolyspora erythraea
tcgtgcgcgcggccctgcgcgcgtagggccgccctgcgggcgtagcgcggccctgcgggcgtagcgcggccctgcgggcttggcgcggcccgggcgggttcagcgcttcgcgcggcgccgcgcgagacggcgcggggccacctgctcggcctgctccccctggatccgcagagccggcggatgtcgttggtgtcgcacgccttcttcaacgccgccctggtcgacgacgacttcgccgccgtcgccaggatctactcgccgatcatcgagaaggcggtcgccgaacagatccgcgaggccgatccggacgccggcgccgagcaggaggcgggaatcctcacctcgctcgtgcgcggcctcatcggcagcgtgctcatcggcgagcggacaccgcagcaggcggtggagctggtggaccggcaactggaccgcgtcttcggcgtcaggagccggtagccgctgacgctcctttcccttcctggcgcgggaagccgcccgctcagccgacctcggcggacagggcgcgcatggtggcgatctcgtcggtctgggtgaccagcacgtcctgggccatcgcgtgcacctgttcgtcgacgccgcgggtgagcaggtcggtcgccatggtcaccgcgccctcgtgatgggcggtcatcagccgcaggaagagccggtcgaagtcggcgccgcgggcggcggccagctcggcgagctgctcgggcgttgccatgcccggcatcgcggcgtgcgcggggtccgcgccggtgtgcccggtgccggtggcgtgcccggtgtccgcgccgccggtgtgcccgccatggccggtgtcgccgccatgcccggtccgcccctgcgcgccgtgggtcgcctgccagccgcgcatcatgtcgatctccggcttctgcgctcccccgatgcgttcggccagcgcccgcacctgcgggtgctgcgcccgctccggggccagggcggtcatctccagcgcctgctcgtggtgcgggatcatcatcgcgacgtaggtcgcttcggcctcgccaggaggtgccggccggccgagcccctggacttcctcgccggtcgcgaccttcggctcgtcgccgggcgcgcccggcaacaccaccggtgcaggcggcggttccggggtcgagcacgcgccgagcagccccgccgcgagaaccaccgcgaacaccgccgccgtcccggtgccgagcctcctcgcggttgcgccgagctgcattgatcctccttataccgacccaaatgcgaccacacggactattggggccgcagaacgtgacaaagatactgattcgggttggtactccggtaccgctgtttggcgagcgcgcgcgcaggcgcgggcagctcgataaccgaatcgaatgtggggtgggttctgttgaatccgagttccaggcgcaggcctggtcgcggcggggcacggttgcgggt

(19) United States
(12) Patent Application Publication
(10) Pub. No.: US 2007/0122885 A1
(43) Pub. Date: May 31, 2007

(54) METHODS OF INCREASING PRODUCTION OF SECONDARY METABOLITES BY MANIPULATING METABOLIC PATHWAYS THAT INCLUDE METHYLMALONYL-COA

(75) Inventors: Andrew Reeves, Chicago, IL (US); J. Mark Weber, Chicago, IL (US); Igor A. Brikun, Forest Park, IL (US)

Correspondence Address: DYKEMA GOSSETT PLLC, 10 S. WACKER DR., STE. 2300, CHICAGO, IL, 60606, US

(73) Assignee: FERMALOGIC, INC., Chicago, IL (US)

(21) Appl. No.: 11/466,364

(22) Filed: Aug. 22, 2006

Related U.S. Application Data
(60) Provisional application No. 60/710,412, filed on Aug. 22, 2005.

(51) Int. Cl. C12P 19/62; C12N 9/10; C12N 15/74; C12N 1/21

(52) U.S. Cl. 435/76; 435/252.35; 435/471; 435/193

(57) ABSTRACT

A process of increasing the cellular production of secondary metabolites, such as antibiotics, is provided. The process is particularly useful for increasing antibiotic production by bacterial cells, especially erythromycin. The process includes the step of increasing the activity of methylmalonyl-CoA mutase.

### CROSS-REFERENCE TO RELATED APPLICATION

This application claims priority to U.S. Provisional Patent Application 60/710,412, filed Aug. 22, 2005, entitled METHODS OF INCREASING PRODUCTION OF BIOLOGICALLY ACTIVE MOLECULES BY MANIPULATING METHYLMALONYL-COA MUTASE, the entirety of which is herein incorporated by reference.

### STATEMENT REGARDING FEDERALLY SPONSORED RESEARCH OR DEVELOPMENT

The subject matter of this application may in part have been funded by the National Institutes of Health, Grant No. R43GM58943, “Antibiotic Regulatory Genes and Metabolic Engineering” and Grant No. R43GM063278-01, “Antibiotic Gene Clusters.” The government may have certain rights in this invention.

### INCORPORATION-BY-REFERENCE OF MATERIAL SUBMITTED ON A COMPACT DISC

Not applicable.

### FIELD OF THE INVENTION

The invention is a process for improving the production of secondary metabolites. When this process is applied to an organism that makes a useful secondary metabolite such as an antibiotic, the organism produces more of the antibiotic.

### BACKGROUND OF THE INVENTION

After a weekend vacation, Alexander Fleming returned to his laboratory to discover that one of his cultures of bacteria had been contaminated with mold. Not only was the plate contaminated, but the bacterial cells, Staphylococcus aureus, had lysed. Instead of throwing the contaminated plates away, Fleming observed that bacterial cell lysis occurred in an area next to the mold and hypothesized that the mold had made a product responsible for the death of the bacteria. He later was able to extract the diffusible substance from the mold, and penicillin was born.

Because antibiotics as a class of drugs are able to kill a broad spectrum of harmful bacterial pathogens, their use has revolutionized medicine, trivializing many diseases that had before taken millions of lives. For example, the plague, caused by infection with the Yersinias pestis bacterium, has laid claim to nearly 200 million lives and has brought about monumental changes, such as the end of the Dark Ages and the advancement of clinical research in medicine. Gentamycin and streptomycin are used to treat patients infected with plague, thus increasing the likelihood of survival. Erythromycins are used to treat respiratory tract and Chlamydia infections, diptheria, Legionnaires' disease, syphilis, anthrax and acne vulgaris. Erythromycins are also used to prevent Streptococcal infections in patients with a history of rheumatic heart disease.

Biological weapons are a real and current threat. Antibiotics are an important defense against the possible devastation such weapons can bring.

Medically important chemical structures made in nature, such as antibiotics, fall into chemical classes based on shared routes of biosynthesis. The macrolides are a group of drugs characterized by the presence of a macrolide ring, a large lactone (a cyclic ester) to which one or more deoxy sugars (in erythromycin the sugars are cladinose and desosamine) are attached. The lactone ring can be either 14, 15 or 16-membered. Macrolides are polyketides, and include erythromycin and its derivatives, such as those marketed as Biaxin®, Rulid®, and Zithromax®.

Erythromycin

Like many secondary metabolites (a metabolite that is produced only under certain physiological conditions), erythromycin is a tailored polymer. The building blocks are one molecule of propionic acid and six molecules of methylmalonic acid in their Coenzyme A (CoA) forms (Omura et al., 1984). Tailoring steps include the addition of two sugars, the addition of a methyl group to one sugar, and the addition of two hydroxyl groups to the polyketide polymer backbone. While the chemical building blocks are known, the source of propionic and methylmalonic acids used to form the molecule are not.

Two sources of these building blocks have been reported: (1) diversion from central metabolic pathways; and (2) amino acid catabolic (break-down) pathways. Evidence for the diversion pathway comes from observations that suggest that succinyl-CoA is the major source of methylmalonyl-CoA via the enzyme methylmalonyl-CoA mutase (MCM) (Hunaiti and Kolattukudy, 1984). Decarboxylation of methylmalonyl-CoA gives rise to propionyl-CoA (Hsieh and Kolattukudy, 1994). These results imply that the precursors for erythromycin biosynthesis are taken at the expense of central metabolism in a reverse-anaplerotic reaction (a reaction that form intermediates of the citric acid cycle). Consistent with these observations, when the mutAB gene is isolated from a rifamycin-producing strain of Amycolatopsis mediterranei U32 and then over-expressed in a monensin (another antibiotic)-producing Streptomyces cinnamonensis host, monensin production increased 32% (Zhang et al., 1999).

Amino acid catabolism has been identified as another source of polyketide precursors (Dotzlaf et al., 1984; Omura et al., 1984; Omura et al., 1983). When branched chain amino acids such as valine, isoleucine, leucine or valine catabolites (propionate and isobutyrate) and threonine are added to fermentation medium, an increase in a macrolide antibiotic and its polyketide-derived precursors is observed (Omura et al., 1984; Omura et al., 1983; Tang et al., 1994). Conversely, when valine catabolism is blocked at the first step (valine dehydrogenase, vdh), production of two different macrolide antibiotics decrease four- to six-fold (Tang et al., 1994). These results suggest that amino acid catabolism, in particular branched-chain amino acid (BCAA) catabolism, is another source of macrolide antibiotic precursors in the Actinomycetes.

Surprisingly, when the branched-chain amino acid catabolic pathway is blocked at a later step in propionyl-CoA carboxylase, macrolide production was not reduced (Donadio et al., 1996; Hunaiti and Kolattukudy, 1984), conflicting with the observations by Dotzlaf et al. (1984). These observations can be explained in part by the use of different macrolide-producing hosts; precursor feeding pathways may not operate universally and be host-dependent.

Methylmalonyl-CoA mutase, encoded by the mutAB gene pair ((Birch et al., 1993; Marsh et al., 1989); see FIG. 7 for a physical map of the region in S. erythraea), is the key enzyme that provides methylmalonyl-CoA for erythromycin biosynthesis (Hunaiti and Kolattukudy, 1984; Zhang et al., 1999). Methylmalonyl-CoA mutase catalyzes the interconversion of methylmalonyl coenzyme A and succinyl coenzyme A; however, succinyl-CoA is favored enzymatically by a factor of twenty to one (Kellermeyer et al., 1964; Vlasie and Banerjee, 2003).

Commercial production of antibiotics, such as erythromycin, is accomplished through large fermentations. However, production is limited to the output that any particular strain is capable of under particular culture conditions. This observation is especially true for secondary products, such as antibiotics, where efficiency and concentrations are both low. To increase efficiency and economy in antibiotic production, strains have been engineered, either by (1) a haphazard, random mutational approach that requires either a selection (rarely available) or laborious, brute-force screens (and some luck), and by directed, or (2) targeted genetic alterations. While the mutational approach is simple to perform and has been successful in generating improved mutants, its ability to provide innovations is limited, and in fact, has not produced any new genetic information in the understanding of strain improvement over the last 60 years. On the other hand, directed genetic manipulation allows not only for strain improvement, but also an understanding of the pathways that produce the antibiotic.

An example of the admirable results of the directed genetic manipulation approach is demonstrated by the targeted knockout of the mutB gene in the model erythromycin-producing Aeromicrobium erythreum bacterium, which resulted in improved antibiotic production (Reeves et al., 2004). The challenge of such results, however, is to transfer the results to a setting that is industry-applicable.

A variable that has recently become a topic of controversy is the use of oils in fermentation media in the culture of Streptomyces cinnamonensis and monensin production, also a secondary metabolite (Li et al., 2004). However, the coupling of genetic manipulation and fermentation condition manipulation to improve and increase polyketide production from a single pathway instead of shifting between pathways has not been heretofore practiced.

### SUMMARY OF THE INVENTION

The invention is directed to methods of increasing polyketide production, especially polyketides, such as erythromycin, by increasing the activity of methylmalonyl-CoA. The invention also includes bacterial cells that have been modified to increase the activity of methylmalonyl-CoA. Finally, the invention is directed to methods of culturing modified cells to increase polyketide production.

### BRIEF DESCRIPTION OF THE DRAWINGS

FIG. 1 shows eythromycin production of S. erythraea wild-type strain FL2267 and mutB mutant FL2281 grown in medium 2 (SCM+5% soybean oil).

FIG. 2 shows erythromycin production of S. erythraea wild type strain FL2267 and mutB mutant FL2281 grown in medium 1 (SCM only).

FIG. 3 shows erythromycin production of S. erythraea wild type strain FL2267 and mutB mutant FL2281 grown in medium 1 and medium 2.

FIG. 4 shows erythromycin production of S. erythraea wild type strain FL2267 and mutB mutant FL2281 grown in medium 1 (SCM only) and medium 3 (SCM+4× starch).

FIG. 5 shows erythromycin production of S. erythraea wild type strain FL2267 and mutB mutant FL2302 grown in medium 1 and medium 2.

FIG. 6 shows erythromycin production of S. erythraea wild type strain FL2267 and mutB mutant FL2302 grown in medium 3 and medium 4 (SCM+⁵% soybean oil+4× starch).

FIG. 7 shows a physical map of the S. erythraea methylmalonyl-CoA mutase region. The entire region sequenced spans 8.6 kb, which includes upstream and downstream sequences. The five ORFs identified in the region are mutA, mutB, gntB, gntR, and SeORF1 (GenBank Accession Nos. DQ289499 and DQ289500 (SEQ ID NOs:12 and 13)) and cover about 6.5 kb. The genes are all transcribed in the same direction, indicated by arrows.

FIG. 8 shows erythromycin production of the S. erythraea mmCoA mutase over-expression strain FL2385. Erythromycin production levels are given as the average of triplicate shake flasks.

### DETAILED DESCRIPTION

The invention is based on the finding that manipulating metabolic pathways that lead to or from a metabolite pool of methylmalonyl CoA within the cell can result in an increase in production of secondary metabolites derived from methylmalonyl CoA. The invention came about because of a striking result that showed that erythromycin production could be increased by increasing the activity of methylmalonyl-CoA mutase, whether directly or indirectly, as well as manipulating culture conditions (Reeves et al., 2006). This result is especially striking when previous results are considered, wherein erythromycin production was increased by decreasing methylmalonyl-CoA mutase activity (Reeves et al., 2004).

Based on these results, the invention exploits the finding and applies it more universally. By increasing the overall concentration of methylmalonyl CoA in the cell, production of important secondary metabolites, including metabolites such as erythromycin, is significantly increased. The methylmalonyl CoA metabolite pool can be increased using a variety of “tools,” which tinker with the input into the pool, as well as with the output. Input is increased by increasing the activity of enzymes, or the concentration of enzymes, that result in the production of methylmamlonyl-CoA. Either simultaneously or alternatively, the output from, or draining of, the methylmalonyl-CoA pool is restricted by decreasing the activity of one or more enzymes that use methylmalonyl-CoA as a substrate, except, for example, the polyketide synthase used in erythromycin biosynthesis.

Several tools in the invention's tool box include various genetic manipulations of the enzymes in pathways that lead to and from the methylmalonyl-CoA pool, as well as culture condition manipulations, notably the choice of carbon source—for example, selecting between carbohydrate and oil. Using the different tools together can produce in some cases optimal results and can be used to “fine-tune” production of the target metabolite.

Aeromicrobium erythreum MCM mutants lacking MCM activity produce about two-fold more erythromycin than the parent strain (Reeves et al., 2004). This technology was transferred to Saccharopolyspora erythraea, the most common, if not universal, industrial erythromycin-producer. Accordingly, an MCM-mutant was generated and tested in shake flask fermentations using standard laboratory medium, soluble complete medium (SCM). As expected, four-fold increase in erythromycin production was observed. mutB mutants also produced as much erythromycin in medium without soybean oil addition (in medium with lower starch concentrations) as the wild-type strains.

However, when the MCM-S. erythraea mutant was cultured in a soy flour-based industrial medium (insoluble production medium) instead of laboratory medium, the mutant unexpectedly produced significantly less erythromycin than the parent strain.

Because the only variable besides the media was the genetic ablation of MCM expression, an MCM over-expression strain was produced and cultured in the two media. This strain had not previously been developed, although a Streptomyces cinnamonensis mutant was produced to over-express an Amycolatopsis mediterranei MCM, resulting in a modest increase in monensin production of 32% in laboratory medium (Zhang et al., 1999). The MCM over-expression mutant increased erythromycin output by 200% in SCM medium and 48% in industrial medium.

Based on these unexpected results, the invention provides for compositions, methods and systems for the improvement of antibiotic production, especially erythromycin.

### DEFINITIONS

SCM means Soluble Complete Medium (McAlpine et al., 1987). A typical formulation appropriate for S. erythraea is per liter: 15 g soluble starch; 20 g Bacto soytone (soybean peptone; Becton-Dickinson); 0.1 g calcium chloride; 1.5 g yeast extract; 10.5 g 3-(N-Morpholino)propanesulfonic acid (MOPS), pH 6.8.

Soy flour is a fine powder made from soybeans (Glycine max).

Unrefined soy source is any form of soybean that can be even partially dissolved in solution, such as SCM or IPM media. “Unrefined” means that the soybean has undergone minimal processing, but does not mean no processing. For example, soy flour is an unrefined soy source. An example of processing includes the production of soybean peptone, such as Bacto soytone.

MCM means the enzyme methylmalonyl-CoA mutase. Any MCM having at least 64% sequence identity to the polynucleotide sequence (SEQ ID NO:8) or polypeptide sequence (SEQ ID NOs:9 and 10) of S. erytheae falls within the scope of the invention. For example, BLAST analysis shows 64% amino acid sequence identity between the mutB polypeptide of A. erythreum and the equivalent human sequence. A high degree of identity exists to all other mutB genes in the database. Also included are those polypeptides having MCM-activity, defined as catalyzing reactants that result in the interconversion of methylmalony-CoA and succinyl-CoA, regardless of the amino acid sequence of the polypeptide.

Regulator means a substance, process, gene, or gene product that controls another substance, process, gene or gene product. A negative regulator is a regulator that decreases another substance, process, gene or gene product; a positive regulator increases another substance, process, gene or gene product.

Complementary refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Nucleic acid fragments are at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full-length sequence. Fragments may be derived from any contiguous portion of a nucleic acid or amino acid sequence of choice.

A homologous nucleic acid sequence or homologous amino acid sequence, or variations thereof, refer to sequences characterized by a homology at the nucleotide level or amino acid level. Homologous nucleotide sequences encode those sequences coding for isoforms of MCM. Isoforms can be expressed in different tissues of the same organism as a result of, for example, alternative splicing of RNA. Alternatively, different genes can encode isoforms. In the invention, homologous nucleotide sequences include nucleotide sequences encoding for a MCM of species other than bacteria, including, but not limited to: vertebrates, and thus can include, e.g., frog, mouse, rat, rabbit, dog, cat, cow, horse, and any organism, including all polyketide-producers. Homologous nucleotide sequences also include, but are not limited to, naturally occurring allelic variations and mutations of the nucleotide sequences set forth herein. A homologous nucleotide sequence does not, however, include the exact nucleotide sequence encoding human MCM. Homologous nucleic acid sequences include those nucleic acid sequences that encode conservative amino acid substitutions in SEQ ID NOs:9 and 10, as well as a polypeptide possessing MCM biological activity.

An open reading frame (ORF) of a MCM gene encodes MCM. An ORF is a nucleotide sequence that has a start codon (ATG) and terminates with one of the three “stop” codons (TAA, TAG, or TGA). In this invention, however, an ORF may be any part of a coding sequence that may or may not comprise a start codon and a stop codon. To achieve a unique sequence, preferable MCM ORFs encode at least 50 amino acids.

Operably linked means a polynucleotide that is in a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous. Enhancers do not have to be contiguous. Linking is accomplished by ligation at convenient restriction sites. If such sites do not exist, the synthetic oligonucleotide adaptors or linkers can be used.

An isolated MCM-encoding polynucleotide is a nucleic acid molecule that is identified and separated from at least one contaminant nucleic acid molecule with which it is ordinarily associated in the natural source of the MCM nucleic acid. An isolated MCM nucleic acid molecule includes those contained in cells that ordinarily express the MCM polypeptide where, for example, the nucleic acid is in a chromosomal location different from that of natural cells, or as provided extra-chromosomally.

An isolated or purified polypeptide, protein or biologically active fragment is separated and/or recovered from a component of its natural environment. Contaminant components include materials that would typically interfere with diagnostic or therapeutic uses for the polypeptide, and may include enzymes, hormones, and other proteinaceous or non-proteinaceous materials. Preferably, the polypeptide is purified to a sufficient degree to obtain at least 15 residues of N-terminal or internal amino acid sequence. To be substantially isolated, preparations having less than 30% by dry weight of non-MCM contaminating material (contaminants), more preferably less than 20%, 10% and most preferably less than 5% contaminants. An isolated, recombinantly-produced MCM or biologically active portion is preferably substantially free of culture medium, i.e., culture medium represents less than 20%, more preferably less than about 10%, and most preferably less than about 5% of the volume of the MCM preparation. Examples of contaminants include cell debris, culture media, and substances used and produced during in vitro synthesis of MCM.

An active MCM polypeptide or MCM polypeptide fragment retains a biological and/or an immunological activity similar, but not necessarily identical, to an activity of a naturally-occurring (wild-type) MCM polypeptide of the invention, including mature forms. A particular biological assay, with or without dose dependency, can be used to determine MCM activity. A nucleic acid fragment encoding a biologically-active portion of MCM can be prepared by isolating a portion of SEQ ID NO:8 that encodes a polypeptide having a MCM biological activity (the biological activities of the MCM are described below), expressing the encoded portion of MCM (e.g., by recombinant expression in vitro) and assessing the activity of the encoded portion of MCM. Immunological activity refers to the ability to induce the production of an antibody against an antigenic epitope possessed by a native MCM; biological activity refers to a function, either inhibitory or stimulatory, caused by a native MCM that excludes immunological activity.

Practicing the Invention

The invention is exemplified by the situation wherein erythromycin production is increased by increasing activity of the MCM, using erythromycin-producing strains to exemplify the methods. Various tools that can be used to manipulate other enzymes that lead to or from the methylmalonyl-CoA metabolite pool are also discussed. Culture conditions are discussed that can be used to maximize antibiotic production, especially using commercial culture conditions.

Increasing methylmalonyl-CoA mutase Activity

In one embodiment, a process of the present invention includes increasing the activity of methylmalonyl-CoA mutase, the enzyme that catalyzes the inter-conversion of methylmalonyl-CoA and succinyl-CoA.

The activity of methylmalonyl-CoA mutase can be increased by any means that results in an increase in production of methylmalonyl-CoA, and ultimately, a polyketide. When increasing the activity of MCM, care should be taken that sufficient substrate and co-factors are available to accommodate the increased activity, including the co-enzyme B12. In some cases, increasing MCM activity simply requires providing additional substrate and co-factors.

The activity of methylmalonyl-CoA mutase (MCM) can also be increased by increasing the amount of enzyme that is expressed. Means of increasing the amount of MCM include: (1) increasing the transcription, translation or copy number of the MCM gene; (2) increasing the transcription, translation, or copy number of a positive regulator of the MCM gene; and (3) decreasing the transcription or translation of a negative regulator of the MCM gene, including genetically inactivating the gene. These approaches can be combined to maximize MCM activity.

Increasing the Transcription, Translation or Copy Number of the MCM Gene or Positive Regulator of the MCM Gene

(a) Control Sequences

One method of increasing transcription is to enlist powerful control sequences. “Control sequences” refers to nucleotide sequences that enable expression of an operably linked coding sequence in a particular host organism. Prokaryotic control sequences include (1) a promoter, (2) optionally an operator sequence, and (3) a ribosome-binding site. Enhancers, which are often separated from the gene of interest, can also be used.

Examples of constitutive promoters include the int promoter of bacteriophage .lambda., the bla promoter of the β-lactamase gene sequence of pBR322, and the promoter of the chloramphenicol acetyl transferase gene sequence of pPR325, and the like. Examples of inducible prokaryotic promoters include the major right and left promoters of bacteriophage λ (PL and PR), the trp, recA, k acZ, λ acI, and gal promoters of E. coli the α-amylase (Ulmanen et al., 1985) and the ζ-28-specific promoters of B. subtilis (Gilman et al., 1984), the promoters of the bacteriophages of Bacillus (Gilman et al., 1984), and Streptomyces promoters (Ward et al., 1986). Prokaryotic promoters are reviewed by (Cenatiempo, 1986); and Gottesman (Gottesman, 1984).

(b) Extra Copies

Another method of increasing MCM activity includes introducing additional copies of an MCM polynucleotide. These extra copies can be extra-chromosomal or integrated into the host organism's genome, or both. Expression from these additional copies can be enhanced using control elements, such as promoters (including inducible promoters), enhancers, etc.. Nucleic acid variants encoding MCM can be used, as well as those that encode polypeptide MCM variants.

Alternatively, additional copies of MCM polynucleotides can be introduced by cross-mating bacteria.

The invention further encompasses using nucleic acid molecules that differ from the nucleotide sequences shown in SEQ ID NO:8 (shown in Table 2; SEQ ID NO:8 shows the MCM operon of S. erythraea; nucleotides 258-2114 encode mutA, the small subunit of MCM; nucleotides 2111-4405 encode mutB, the large subunit of MCM; nucleotides 4408-5394 encode meaB; and nucleotides 5394-5753 encode gntR) due to degeneracy of the genetic code and thus encode the same MCM as that encoded by the nucleotide sequences shown in SEQ ID NO:8. An isolated nucleic acid molecule useful in the invention has a nucleotide sequence encoding proteins, among others, having amino acid sequences shown in SEQ ID NOs:9 and 10 (shown in Table 1).

Table 3 shows SEQ ID NOs:12 and 13, wherein SEQ ID NO:12 represents the genomic sequences that are upstream of mutA, and includes ORFSe1 from nucleotide 236 to 1147. In SEQ ID NO:13, showing the genomic sequence downstream of gntR, encodes from nucleotide 500-1234, ORFSe6, a protein that is similar to putative lipoproteins in Streptomyces coelicolor and Streptomyces avermitilis.

TABLE 1

Methylmalonyl CoA operon - encoded polypeptides
(SEQ ID NOs: 7, 9, 10 and 11)

```
mutA (SEQ ID NO:9)
Met Ala His Ser Thr Thr Ser Asp Gly Pro Glu Leu Pro Leu Ala Ala
1 5 10 15
Glu Phe Pro Glu Pro Ala Arg Gln Gln Trp Arg Gln Gln Val Glu Lys
                        20 25 30
Val Leu Arg Arg Ser Gly Leu Leu Pro Glu Gly Arg Pro Ala Pro Glu
                35 40 45
Pro Val Glu Asp Val Leu Ala Ser Ala Thr Tyr Asp Gly Ile Thr Val
        50 55 60
His Pro Leu Tyr Thr Glu Gly Pro Ala Ser Ser Gly Val Pro Gly Leu
65 70 75 80
Ala Pro Tyr Val Arg Gly Ser Arg Ala Gln Gly Cys Val Ser Glu Gly
                                85 90 95
Trp Asp Val Arg Gln His His Ala His Pro Asp Ala Ser Glu Thr Asn
                        100 105 110
Arg Glu Ile Leu Ala Asp Leu Tyr Asn Gly Thr Thr Ser Leu Trp Leu
                115 120 125
Glu Leu Gly Pro Thr Gly Leu Pro Val Asp Ser Leu Ala Asp Ala Leu
        130 135 140
Glu Gly Val His Leu Asp Met Ile Gly Val Val Leu Asp Ala Gly Asp
145 150 155 160
Glu Ala Ala Arg Ala Ala Ser Ala Leu Leu Glu Leu Ala Arg Glu Gln
                                165 170 175
Gly Val Arg Pro Ser Ala Leu Arg Ala Asn Leu Gly Ala Asp Pro Leu
                        180 185 190
Ser Thr Trp Ala Arg Thr Gly Gln Glu Arg Asp Leu Gly Leu Ala Ala
                195 200 205
Glu Val Ala Ala His Cys Ala Ser His Pro Gly Leu Arg Ala Ile Thr
        210 215 220
Val Asp Gly Leu Pro Tyr His Glu Ala Gly Gly Ser Asp Ala Glu Glu
225 230 235 240
Leu Gly Cys Ser Ile Ala Ala Gly Val Thr Tyr Leu Arg Val Leu Ala
                                245 250 255
Gly Glu Leu Gly Ala Glu Ala Ala Ser Gly Leu Leu Glu Phe Arg Tyr
                        260 265 270
Ala Ala Thr Ala Asp Gln Phe Leu Thr Ile Ala Lys Leu Arg Ala Ala
                275 280 285
Arg Arg Leu Trp Glu Arg Val Thr Arg Glu Ile Gly Val Ala Glu Arg
        290 295 300
Ala Gln Leu Gln His Ala Val Thr Ser Ser Ala Met Leu Thr Arg Arg
305 310 315 320
Asp Pro Trp Val Asn Met Leu Arg Thr Thr Ile Ala Thr Phe Ala Ala
                                325 330 335
Gly Val Gly Gly Ala Arg Ser Val Thr Val Arg Pro Phe Asp Ala Ala
                        340 345 350
Ile Gly Leu Pro Asp Pro Phe Ser Arg Arg Ile Ala Arg Asn Thr Gln
                355 360 365
Ser Leu Leu Leu Glu Glu Ser His Leu Ala Gln Val Ile Asp Pro Ala
        370 375 380
Gly Gly Ser Trp Tyr Val Glu Thr Leu Thr Asp Glu Leu Ala His Lys
385 390 395 400
Ala Trp Glu Trp Phe Arg Arg Ile Glu Ala Glu Gly Gly Leu Pro Ala
                                405 410 415
Ala Leu Arg Ser Gly Leu Val Ala Asp Arg Leu Ala Glu Thr Trp Gln
                        420 425 430
Arg Arg Arg Asp Ala Val Ala His Arg Thr Asp Pro Ile Thr Gly Val
                435 440 445
Thr Glu Phe Pro Asn Leu Glu Glu Pro Ala Leu Arg Arg Asp Pro Ala
        450 455 460
Pro Glu Pro Leu Ser Gly Gly Leu Pro Arg His Arg Tyr Ala Glu Asp
465 470 475 480
Phe Glu Arg Leu Arg Asp Ala Ser Asp Ala His Leu Ala Glu Thr Gly
                                485 490 495
Ala Arg Pro Lys Val Phe Leu Ala Thr Leu Gly Ser Leu Ala Glu His
                        500 505 510
Asn Ala Arg Ala Ser Phe Ala Arg Asn Leu Phe Gly Ala Gly Gly Leu
                515 520 525
Glu Thr Pro Asp Ala Gly Pro Thr Glu Ser Thr Glu Asp Val Val Lys
        530 535 540
Ala Phe Ala Gly Ser Gly Thr Pro Val Ala Cys Leu Cys Ser Gly Asp
545 550 555 560
Arg Ile Tyr Gly Glu His Ala Glu Glu Thr Ala Arg Ala Leu Arg Glu
                                565 570 575
Ala Gly Ala Asp Gln Val Leu Leu Ala Gly Ser Leu Glu Val Pro Gly
                        580 585 590
Val Asp Gly Arg Val Phe Gly Gly Cys Asn Ala Leu Glu Val Leu Gln
                595 600 605
Asp Val His Arg Arg Leu Gly Val Gln Gln
        610 615
mutB (SEQ ID NO:10)
Met Thr Ala His Glu His Glu Pro Ile Pro Ser Phe Ala Gly Val Glu
1 5 10 15
Leu Gly Glu Pro Ala Pro Ala Pro Ala Gly Arg Trp Asn Asp Ala Leu
                        20 25 30
Leu Ala Glu Thr Gly Lys Glu Ala Asp Ala Leu Val Trp Glu Ala Pro
                35 40 45
Glu Gly Ile Gly Val Lys Pro Leu Tyr Thr Glu Ala Asp Thr Arg Gly
        50 55 60
Leu Asp Phe Leu Arg Thr Tyr Pro Gly Ile Ala Pro Phe Leu Arg Gly
65 70 75 80
Pro Tyr Pro Thr Met Tyr Val Asn Gln Pro Trp Thr Val Arg Gln Tyr
                                85 90 95
Ala Gly Phe Ser Thr Ala Glu Gln Ser Asn Ala Phe Tyr Arg Arg Asn
                        100 105 110
Leu Ala Ala Gly Gln Lys Gly Leu Ser Val Ala Phe Asp Leu Ala Thr
                115 120 125
His Arg Gly Tyr Asp Ser Asp His Pro Arg Val Gly Gly Asp Val Gly
        130 135 140
Met Ala Gly Val Ala Ile Asp Ser Ile Tyr Asp Met Arg Arg Leu Phe
145 150 155 160
Asp Gly Ile Pro Leu Asp Arg Met Ser Val Ser Met Thr Met Asn Gly
                                165 170 175
Ala Val Leu Pro Val Met Ala Leu Tyr Ile Val Ala Ala Glu Glu Gln
                        180 185 190
Gly Val Ala Pro Glu Lys Leu Ala Gly Thr Ile Gln Asn Asp Ile Leu
                195 200 205
Lys Glu Phe Met Val Arg Asn Thr Tyr Ile Tyr Pro Pro Gln Pro Ser
        210 215 220
Met Arg Ile Ile Ser Asp Ile Phe Ala Tyr Ala Ser Arg Arg Met Pro
225 230 235 240
Lys Phe Asn Ser Ile Ser Ile Ser Gly Tyr His Ile Gln Glu Ala Gly
                                245 250 255
Ala Thr Ala Asp Leu Glu Leu Ala Tyr Thr Leu Ala Asp Gly Val Glu
                        260 265 270
Tyr Leu Arg Ala Gly Arg Gln Ala Gly Leu Asp Ile Asp Ser Phe Ala
                275 280 285
Pro Arg Leu Ser Phe Phe Trp Gly Ile Gly Met Asn Phe Ala Met Glu
        290 295 300
Val Ala Lys Leu Arg Ala Ala Arg Leu Leu Trp Ala Lys Leu Val Lys
305 310 315 320
Arg Phe Glu Pro Ser Asp Pro Lys Ser Leu Ser Leu Arg Thr His Ser
                                325 330 335
Gln Thr Ser Gly Trp Ser Leu Thr Ala Gln Asp Val Tyr Asn Asn Val
                        340 345 350
Val Arg Thr Cys Val Glu Ala Met Ala Ala Thr Gln Gly His Thr Gln
                355 360 365
Ser Leu His Thr Asn Ala Leu Asp Glu Ala Leu Ala Leu Pro Thr Asp
        370 375 380
Phe Ser Ala Arg Ile Ala Arg Asn Thr Gln Leu Val Leu Gln Gln Glu
385 390 395 400
Ser Gly Thr Thr Arg Val Ile Asp Pro Trp Gly Gly Ser His Tyr Ile
                                405 410 415
Glu Arg Leu Thr Gln Asp Leu Ala Glu Arg Ala Trp Ala His Ile Thr
                        420 425 430
Glu Val Glu Asp Ala Gly Gly Met Ala Gln Ala Ile Asp Ala Gly Ile
                435 440 445
Pro Lys Met Arg Ile Glu Glu Ala Ala Ala Arg Thr Gln Ala Arg Ile
        450 455 460
Asp Ser Gly Arg Gln Pro Leu Ile Gly Val Asn Lys Tyr Arg Tyr Asp
465 470 475 480
Gly Asp Glu Gln Ile Glu Val Leu Lys Val Asp Asn Ala Gly Val Arg
                                485 490 495
Ala Gln Gln Leu Asp Lys Leu Arg Arg Leu Arg Glu Glu Arg Asp Ser
                        500 505 510
Glu Ala Cys Glu Thr Ala Leu Arg Arg Leu Thr Gly Ala Ala Glu Ala
                515 520 525
Ala Leu Glu Asp Asn Arg Pro Asp Asp Leu Ala His Asn Leu Leu Thr
        530 535 540
Leu Ala Val Asp Ala Ala Arg His Lys Ala Thr Val Gly Glu Ile Ser
545 550 555 560
Asp Ala Leu Glu Lys Val Phe Gly Arg His Ser Gly Gln Ile Arg Thr
                                565 570 575
Ile Ser Gly Val Tyr Arg Glu Glu Ser Gly Thr Ser Glu Ser Leu Glu
                        580 585 590
Arg Ala Arg Arg Lys Val Glu Glu Phe Asp Glu Ala Glu Gly Arg Arg
                595 600 605
Pro Arg Ile Leu Val Ala Lys Met Gly Gln Asp Gly His Asp Arg Gly
        610 615 620
Gln Lys Val Ile Ala Thr Ala Phe Ala Asp Ile Gly Phe Asp Val Asp
625 630 635 640
Val Gly Pro Leu Phe Gln Thr Pro Ala Glu Val Ala Arg Gln Ala Val
                                645 650 655
Glu Ser Asp Val His Val Val Gly Val Ser Ser Leu Ala Ala Gly His
                        660 665 670
Leu Thr Leu Val Pro Ala Leu Arg Asp Glu Leu Ala Gly Leu Gly Arg
                675 680 685
Ser Asp Ile Met Ile Val Val Gly Gly Val Ile Pro Pro Ala Asp Phe
        690 695 700
Asp Ala Leu Arg Gln Gly Gly Ala Ser Ala Ile Phe Pro Pro Gly Thr
705 710 715 720
Val Ile Ala Asp Ala Ala Leu Gly Leu Leu Asp Gln Leu Arg Ala Val
                                725 730 735
Leu Asp His Pro Ala Pro Gly Glu Pro Ala Gly Glu Ser Asp Gly Ala
                        740 745 750
Arg Gly Gly Ser Pro Gly Glu Thr Ser Ser Ala Gly
                755 760
meaB (SEQ ID NO:8)
Met Pro Arg Glu Ile Asp Val Gln Asp Tyr Ala Lys Gly Val Leu Gly
1 5 10 15
Gly Ser Arg Ala Lys Leu Ala Gln Ala Ile Thr Leu Val Glu Ser Thr
                        20 25 30
Arg Ala Glu His Arg Ala Lys Ala Gln Glu Leu Leu Val Glu Leu Leu
                35 40 45
Pro His Ser Gly Gly Ala His Arg Val Gly Ile Thr Gly Val Pro Gly
        50 55 60
Val Gly Lys Ser Thr Phe Ile Glu Ser Leu Gly Thr Met Leu Thr Ala
65 70 75 80
Gln Gly His Arg Val Ala Val Leu Ala Val Asp Pro Ser Ser Thr Arg
                                85 90 95
Ser Gly Gly Ser Ile Leu Gly Asp Lys Thr Arg Met Pro Lys Phe Ala
                        100 105 110
Ser Asp Ser Gly Ala Phe Val Arg Pro Ser Pro Ser Ala Gly Thr Leu
                115 120 125
Gly Gly Val Ala Arg Ala Thr Arg Glu Thr Ile Val Leu Met Glu Ala
        130 135 140
Ala Gly Phe Asp Val Val Leu Val Glu Thr Val Gly Val Gly Gln Ser
145 150 155 160
Glu Val Ala Val Ala Gly Met Val Asp Cys Phe Leu Leu Leu Thr Leu
                                165 170 175
Ala Arg Thr Gly Asp Gln Leu Gln Gly Ile Lys Lys Gly Val Leu Glu
                        180 185 190
Leu Ala Asp Leu Val Ala Val Asn Lys Ala Asp Gly Pro His Glu Gly
                195 200 205
Glu Ala Arg Lys Ala Ala Arg Glu Leu Arg Gly Ala Leu Arg Leu Leu
        210 215 220
Thr Pro Val Ser Thr Ser Trp Arg Pro Pro Val Val Thr Cys Ser Gly
225 230 235 240
Leu Thr Gly Ala Gly Leu Asp Thr Leu Trp Glu Gln Val Glu Gln His
                                245 250 255
Arg Ala Thr Leu Thr Glu Thr Gly Glu Leu Ala Glu Lys Arg Ser Arg
                        260 265 270
Gln Gln Val Asp Trp Thr Trp Ala Leu Val Arg Asp Gln Leu Met Ser
                275 280 285
Asp Leu Thr Arg His Pro Ala Val Arg Arg Ile Val Asp Glu Val Glu
        290 295 300
Ser Asp Val Arg Ala Gly Glu Leu Thr Ala Gly Ile Ala Ala Glu Arg
305 310 315 320
Leu Leu Asp Ala Phe Arg Glu Arg
                                325
gntR (SEQ ID NO:11)
Met Leu Ala Val Thr Val Asp Pro Asn Ser Ala Val Ala Pro Phe Glu
1 5 10 15
Gln Val Arg Thr Gln Ile Ala Gln Gln Ile Asn Asp Arg Val Leu Pro
                        20 25 30
Val Gly Thr Lys Leu Pro Thr Val Arg Arg Leu Ala Ala Asp Leu Gly
                35 40 45
Ile Ala Ala Asn Thr Ala Ala Lys Ala Tyr Arg Glu Leu Glu Gln Ala
        50 55 60
Gly Leu Ile Glu Thr Arg Gly Arg Ala Gly Thr Phe Val Gly Ser Ala
65 70 75 80
Gly Glu Arg Ser Asn Glu Arg Ala Ala Glu Ala Ala Ala Glu Tyr Ala
                                85 90 95
Arg Thr Val Ala Ala Leu Gly Ile Pro Arg Glu Glu Ala Leu Ala Ile
                        100 105 110
Val Arg Ala Ala Leu Arg Ala
                115
```

TABLE 2
MCM operon GenBank Accession No. AY117133. (SEQ ID NO:8)

| | |
|---|---|
| ggttctcgga gtcggcggtc ccggtgcggt gcaggcggct gcgccaaggc gcaccggctg | 60 |
| ccgggcgcgg gaccgacgag ctgacactgg tgggtggtcg ttcggtgcac ctcgcggtgc | 120 |
| gggacgtccc gcgcggcgtg ctcgggatcg cctgggactg ggactgaggc gcccggcgga | 180 |
| cgctctgccc tgtccggctg cgacaagcgt cacacgatcc ccgggccggg ccgcaccggc | 240 |
| ctaccatcct gttcatggtg gcgcactcga cgacgagcga cgggccggag ctgcccctgg | 300 |
| cggccgagtt ccccgagccc gcccggcagc agtggcggca acaggtggag aaggtcctgc | 360 |
| gcaggtcggg tctgctgccc gagggcaggc ccgcgccgga gccggtcgag gacgtgctcg | 420 |
| ccagcgccac ctacgacggc atcaccgtgc acccgctcta caccgagggt cccgcatcca | 480 |
| gcggcgtccc gggcctggcg ccctacgtgc gcggcagccg ggcgcagggc tgcgtcagcg | 540 |
| agggctggga cgtccgccag caccacgccc accccgacgc ctcggagacc aaccgcgaga | 600 |
| tcctggccga cctctacaac ggcacgacct cgctgtggct ggagctcggg ccgaccgggc | 660 |
| tgccggtgga ctcgctggcc gacgccctcg aaggcgtcca cctggacatg atcggcgtcg | 720 |
| tgctcgacgc cggtgacgag gcggcgcggg ccgcgtcggc gttgctggag ctcgcgcggg | 780 |
| agcagggggt gcggcccagc gcgctgcgcg ccaacctggg cgccgacccg ctgagcacct | 840 |
| gggctcgcac cgggcaggaa cgcgacctgg gcctcgccgc cgaggtcgcc gcgcactgcg | 900 |
| cgtcgcaccc gggcctgcgc gcgatcaccg tcgacggcct gccctaccac gaggcgggcg | 960 |
| gctccgacgc cgaggagctc ggctgctcga tcgccgcggg cgtcacctac ctgcgggtgc | 1020 |
| tggccggtga gctcggtgcc gaggccgcga gcgggctgct ggagttccgc tacgccgcca | 1080 |
| ccgccgacca gttcctgacc atcgccaagc tgcgcgcggc ccgcaggctg tgggagcggg | 1140 |
| tgacgcggga gatcggcgtc gccgagcgcg cgcagctcca gcacgcggtc acctcctcgg | 1200 |
| cgatgctgac gcgccgcgac ccgtgggtga acatgctgcg caccacgatc gccacgttcg | 1260 |
| ccgcaggcgt gggcggcgcg cggtcggtca ccgtgcgccc gttcgacgcc gcgatcgggc | 1320 |
| tgccggaccc cttctcccgg cgcatcgccc gcaacaccca gtcgctgctg ctggaggagt | 1380 |
| cgcacctggc gcaggtgatc gacccggcgg gcggttcctg gtacgtcgag acgctgaccg | 1440 |
| acgaactggc gcacaaggcg tgggagtggt tccggcgcat cgaggccgag ggcgggctgc | 1500 |
| ccgccgcgct gcgctcgggt ctggtggccg accggctcgc cgagacctgg cagcggcgcc | 1560 |
| gggacgccgt cgcccaccgc accgacccga tcaccggcgt caccgagttc ccgaacctcg | 1620 |
| aagaacccgc gctgcgacgc gaccccgcgc ccgagccgct gtcgggcggc ctgccccgcc | 1680 |
| accgctacgc cgaggacttc gagcggctgc gcgacgcctc cgacgcccac ctcgccgaaa | 1740 |
| ccggtgcgcg cccgaaggtc ttcctcgcca cgctcggttc gctcgccgag cacaacgccc | 1800 |
| gcgcgtcgtt cgcccgcaac ctcttcggcg cgggcgggct ggaaaccccg gacgccgggc | 1860 |
| ccacggagtc cacagaggac gtggtgaagg cgttcgccgg ctcgggcacg ccggtggcct | 1920 |
| gcctgtgctc gggtgaccgg atctacggtg agcacgcgga ggaaaccgcc cgcgcgctcc | 1980 |
| gggaggcggg ggccgaccag gtgctgctgg ccggctcgct cgaggtgccc ggcgtcgacg | 2040 |
| gccgggtgtt cggcgggtgc aacgccctcg aagtcttgca ggacgtccac cgcaggttgg | 2100 |
| gagtgcagca gtgaccgccc acgagcacga accgatcccc agcttcgccg gcgtggagct | 2160 |
| gggcgagccc gcccccgcgc ctgccgggcg gtggaacgac gcgctgctgg ccgagaccgg | 2220 |
| caaggaggcc gacgccctgg tgtgggaggc gcccgagggc atcggcgtca agccgctcta | 2280 |
| caccgaggcc gacacccgcg ggctggactt cctgcgcacc tacccgggaa tcgcgccgtt | 2340 |
| cctgcgcggc ccgtacccga cgatgtatgt caaccagccg tggacggtgc gccagtacgc | 2400 |
| ggggttctcc accgccgagc agtccaacgc cttctaccgc cgcaacctcg ccgccgggca | 2460 |
| gaagggcctg tcggtggcct tcgacctggc cacccaccgc ggctacgact ccgaccaccc | 2520 |
| gcgcgtcggc ggtgacgtcg gcatggcggg cgtggcgatc gactccatct atgacatgcg | 2580 |
| ccggctcttc gacggcatcc cgctggacag gatgagcgtg tcgatgacga tgaacggcgc | 2640 |
| cgtgctgccg gtgatggcgc tctacatcgt cgccgccgag gaacagggcg tggcgccgga | 2700 |
| gaagctggcc gggaccatcc agaacgacat cctcaaggag ttcatggtcc gcaacaccta | 2760 |
| catctacccg ccgcagccgt cgatgcggat catctccgac atcttcgcct acgcctcgcg | 2820 |
| gcggatgccg aagttcaact cgatctccat ctccggctac cacatccagg aggccggggc | 2880 |
| gaccgccgac ctggagctgg cctacaccct cgcggacggc gtggagtacc tgcgcgccgg | 2940 |
| gcggcaggcg ggcctggaca tcgactcctt cgccccgcgg ctgtcgttct tctggggcat | 3000 |
| cgggatgaac ttcgcgatgg aggtcgccaa gctgcgcgcg gcccggctgc tgtgggccaa | 3060 |
| gctggtcaag cgcttcgagc cgtcggaccc gaagtcgctg tcgctgcgca cccactcgca | 3120 |
| gacctcgggc tggtcgctga ccgcccagga cgtctacaac aacgtcgtgc gcacgtgcgt | 3180 |
| ggaggcgatg gccgccaccc agggccacac ccagtcgctg cacaccaacg ccctggacga | 3240 |
| ggcgctggcg ctgccgaccg acttctccgc gcgcatcgcc cgcaacaccc agctggtgct | 3300 |
| ccagcaggag tccggcacca cccgcgtcat cgacccgtgg ggcggctcgc actacatcga | 3360 |
| gcggctgacc caggacctcg ccgaacgcgc gtgggcccac atcaccgagg tcgaggacgc | 3420 |
| cggcggcatg gcccaggcca tcgacgccgg tatcccgaag atgcgcatcg aggaggccgc | 3480 |
| cgcgcggacg caggcgcgca tcgactccgg ccgccagccg ctcatcggcg tcaacaagta | 3540 |
| ccgctacgac ggcgacgagc agatcgaggt cctcaaggtc gacaacgccg gcgtgcgggc | 3600 |
| ccagcagctg gacaagctgc ggcggctgcg cgaggaacgc gactccgagg cgtgcgagac | 3660 |
| cgcactgcgc aggctgaccg gcgccgccga ggccgcgctg gaggacaacc ggcccgacga | 3720 |
| cctcgcgcac aacctgctga cgctggccgt ggacgccgcg cggcacaagg ccaccgtcgg | 3780 |
| cgagatctcc gacgcgctgg agaaggtctt cggccgccac tccggccaga tccgtacgat | 3840 |
| ttccggcgtg taccgggagg agtcgggtac ctcggagtcg ctggagcgcg cccgccgcaa | 3900 |
| ggtcgaggag ttcgacgagg cagagggcag gcgcccgcgc atcctggtgg ccaagatggg | 3960 |
| ccaggacggc cacgaccgcg gccagaaggt catcgccacc gccttcgccg acatcggctt | 4020 |
| cgacgtcgac gtgggcccgc tgttccagac cccggccgag gtcgcccgcc aggcggtcga | 4080 |
| gtccgacgtg cacgtcgtcg gggtgtcgtc gctggccgcg ggccacctga cgctggtgcc | 4140 |
| cgcgctgcgc gacgagctgg ccgggctcgg ccgctccgac atcatgatcg ttgtcggcgg | 4200 |
| cgtgatcccg cccgccgact tcgacgcgct gcgccagggc ggagccagcg cgatcttccc | 4260 |
| gccgggaacc gtgatcgccg acgccgcgct cggactgctc gaccagctcc gcgcggtgct | 4320 |
| cgaccacccc gcgcccggcg agcctgccgg cgagtcggac ggcgcccgag gcggttcccc | 4380 |
| cggcgagacg tcgagcgcgg gctgaccatg ccgcgcgaga tcgacgtcca ggactacgcc | 4440 |
| aagggcgtgc tcggcggctc gcgcgccaag ctggcgcagg cgatcacgct ggtggagtcg | 4500 |
| accagggccg agcaccgcgc gaaagcccag gaactgctcg tcgagctgct gccgcacagc | 4560 |
| ggtggggcgc accgggtggg catcaccggc gtgcccggcg tcggcaagtc gacgttcatc | 4620 |
| gagtcgctgg gcacgatgct gaccgcgcag gggcaccggg tcgcggtgct ggcggtcgac | 4680 |
| ccgtcgtcca cgcgcagcgg cggcagcatc ttgggcgaca agacgcggat gcccaagttc | 4740 |
| gcctccgact ccggcgcgtt cgtgcggccc tccccctcgg cgggcacgct cggcggcgtc | 4800 |
| gcgcgcgcga cccgcgagac gatcgtgctg atggaggcgg ccggattcga cgtcgtgctc | 4860 |
| gtggaaacgg tgggcgtcgg ccagtccgag gtcgccgtgg cgggaatggt cgactgcttc | 4920 |
| ctgctgctga cgctggcccg caccggcgac cagttgcagg gcatcaagaa gggtgtgttg | 4980 |
| gagctggccg accttgtcgc ggtgaacaag gccgacggac cgcacgaggg cgaggcgcgc | 5040 |
| aaggcggccc gcgagctgcg cggcgcgctg cggctgctga ccccggtcag cacgtcgtgg | 5100 |
| agacccccgg tggtgacctg cagcggcctg accggagcgg gcctggacac gctctgggag | 5160 |
| caggtcgagc agcaccgcgc caccctcacc gagaccggcg agctggccga gaagcgcagc | 5220 |
| cgccagcagg tcgactggac ctgggcgctg gtgcgcgacc agctcatgtc cgacctgacc | 5280 |
| cggcacccgg cggtgcgccg catcgtcgac gaggtcgaat ccgacgtgcg ggccggggaa | 5340 |
| ctgaccgcgg gcatcgccgc cgagcggctg ctcgacgcct tccgggagcg ctgatgctgg | 5400 |
| ccgtcaccgt cgaccccaac tccgctgtcg caccgttcga gcaggtgcgc acgcagatcg | 5460 |
| cgcagcagat caacgaccgc gtcctgccgg tcggaaccaa gctgcccacc gtgcgccggc | 5520 |
| tggcggccga cctcggcatc gcggccaaca ccgcggccaa ggcctaccgc gagctggagc | 5580 |
| aggcgggact gatcgaaacc cgtggccgcg cgggaacctt cgtgggctcg gcgggcgagc | 5640 |
| gcagcaacga gcgcgcggcc gaggccgccg ccgagtacgc ccggaccgtc gccgcgctgg | 5700 |
| gcatcccccg cgaggaggca cttgccatcg tgcgcgcggc cctgcgcgcg tagggccgcc | 5760 |
| ctgcgggcgt agcgcggccc tgcgggcgta gcgcggccct gcgggcttgg cgcggcccgg | 5820 |
| gcgggttcag cgcttcgcgc ggcgccgcgc gagacggcgc ggggccacct gctcggcctg | 5880 |
| ctccccctgg atcc | 5894 |

TABLE 3

SeORF1, mutA, mutB, meaB, and gntR genes (GenBank Accession Nos.
DQ289499 and DQ289500 (SEQ ID NOs:12 and 13)

| | |
|---|---|
| SEQ ID NO:12 | |
| ccatcgtgcc gcccatcgtg cacggctgcc gcgaaccggc gcggagcagc cgcgataccg | 60 |
| cgcggcgaag ccgaatccga catgttcgca ctccgcgcgc gtgcgcggca ccgccgtgca | 120 |
| acggtgaatt caccagccga gcggctgtgt cgcgcggacc ggcggcggcc atagcctggc | 180 |
| cgcgggcgca cgatccgctg cgcgccaggg agaaccgcgc gctacggagg tcgccatgtc | 240 |
| cggccacggc caatcggacg gcaccgcgtc gagccggccg tgcgaggact cccgcgccga | 300 |
| ggtggaggcc ctgctgcggt ccggtccctt ccacgaggcg ctgcgcgcgg ccatcgcgca | 360 |
| cagcggactc accctggagg ccctgcgcgg tgaactggcc gcgcgcggca tccggctcag | 420 |
| cctggcgacc ctgagctact ggcagcacgg gcgaagccgc cccgagcgga ccggctcgat | 480 |
| gctggcgctg cgcgcgatcg agaacatcct gcggctgccc gcgcattcgc tgcgcgcgct | 540 |
| gctgggtccg ccgcgcccgc gcggccggtg gctcaaccac gagcccggcc gcggcatcga | 600 |
| cgaccccgcc gggcagctcg cggaggtgat cgggccggtg ctggggccgt ccgaccgcga | 660 |
| cctgcgcgtc ttctcccagg aggacatcgc ctccgtcggc ccggaccggg cgatccacct | 720 |
| ggtgcgtacc cgcacggtgc tgcgcgcgct ggccgacggg cccgaccgcc acctcgccgt | 780 |
| ctaccgcggc gaacccggca ccgactcggg cgcgctggtc ccggtcgcca ccgagaactg | 840 |
| ccggctcggc cggaccagca ggcacccggc cgccccgatc gtggtcgccg agctgttgtt | 900 |
| cgaccgcagg atgcgcgccg gggagaccca cctgctggag tacgagttcc gcgtcgagcg | 960 |
| cccggtgcgc agcgtcgacc accgccgcac gttccggtac ccggcgggca gctacgtcgc | 1020 |
| gtcggtgcgg ttctcggagt cggcggtccc ggtgcggtgc aggcggctgc gccaaggcgc | 1080 |
| accggctgcc gggcgcggga ccgacgagct gacactggtg ggtggtcgtt cggtgcacct | 1140 |
| cgcggtg | 1147 |
| SEQ ID NO:13 | |
| tcgtgcgcgc ggccctgcgc gcgtagggcc gccctgcggg cgtagcgcgg ccctgcgggc | 60 |
| gtagcgcggc cctgcgggct tggcgcggcc cgggcgggtt cagcgcttcg cgcggcgccg | 120 |
| cgcgagacgg cgcggggcca cctgctcggc ctgctccccc tggatccgca gagccggcgg | 180 |
| atgtcgttgg tgtcgcacgc cttcttcaac gccgccctgg tcgacgacga cttcgccgcc | 240 |
| gtcgccagga tctactcgcc gatcatcgag aaggcggtcg ccgaacagat ccgcgaggcc | 300 |
| gatccggacg ccggcgccga gcaggaggcg ggaatcctca cctcgctcgt gcgcggcctc | 360 |
| atcggcagcg tgctcatcgg cgagcggaca ccgcagcagg cggtggagct ggtggaccgg | 420 |
| caactggacc gcgtcttcgg cgtcaggagc cggtagccgc tgacgctcct ttcccttcct | 480 |
| ggcgcgggaa gccgcccgct cagccgacct cggcggacag ggcgcgcatg gtggcgatct | 540 |
| cgtcggtctg ggtgaccagc acgtcctggg ccatcgcgtg cacctgttcg tcgacgccgc | 600 |
| gggtgagcag gtcggtcgcc atggtcaccg cgccctcgtg atgggcggtc atcagccgca | 660 |
| ggaagagccg gtcgaagtcg gcgccgcggg cggcggccag ctcggcgagc tgctcgggcg | 720 |
| ttgccatgcc cggcatcgcg gcgtgcgcgg ggtccgcgcc ggtgtgcccg gtgccggtgg | 780 |
| cgtgcccggt gtccgcgccg ccggtgtgcc cgccatggcc ggtgtcgccg ccatgcccgg | 840 |
| tccgcccctg cgcgccgtgg gtcgcctgcc agccgcgcat catgtcgatc tccggcttct | 900 |
| gcgctccccc gatgcgttcg gccagcgccc gcacctgcgg gtgctgcgcc cgctccgggg | 960 |
| ccagggcggt catctccagc gcctgctcgt ggtgcgggat catcatcgcg acgtaggtcg | 1020 |
| cttcggcctc gccaggaggt gccggccggc cgagcccctg gacttcctcg ccggtcgcga | 1080 |
| ccttcggctc gtcgccgggc gcgcccggca acaccaccgg tgcaggcggc ggttccgggg | 1140 |
| tcgagcacgc gccgagcagc cccgccgcga gaaccaccgc gaacaccgcc gccgtcccgg | 1200 |
| tgccgagcct cctcgcggtt gcgccgagct gcattgatcc tccttatacc gacccaaatg | 1260 |
| cgaccacacg gactattggg gccgcagaac gtgacaaaga tactgattcg ggttggtact | 1320 |
| ccggtaccgc tgtttggcga gcgcgcgcgc aggcgcgggc agctcgataa ccgaatcgaa | 1380 |
| tgtggggtgg gttctgttga atccgagttc caggcgcagg cctggtcgcg gcggggcacg | 1440 |
| gttgcgggt | 1449 |

Moreover, MCM from other species that have a nucleotide sequence that differs from the sequence of SEQ ID NO:8, are contemplated. Nucleic acid molecules corresponding to natural allelic variants and homologues of the MCM cDNAs of the invention can be isolated based on their homology to the MCM of SEQ ID NO:8 using cDNA-derived probes to hybridize to homologous MCM sequences under stringent conditions.

“MCM variant polynucleotide” or “MCM variant nucleic acid sequence” means a nucleic acid molecule which encodes an active MCM that (1) has at least about 80% nucleic acid sequence identity with a nucleotide acid sequence encoding a full-length native MCM, (2) a full-length native MCM lacking the signal peptide, (3) an extracellular domain of a MCM, with or without the signal peptide, or (4) any other fragment of a full-length MCM. Ordinarily, a MCM variant polynucleotide will have at least about 60% nucleic acid sequence identity, more preferably at least about 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% nucleic acid sequence identity and yet more preferably at least about 99% nucleic acid sequence identity with the nucleic acid sequence encoding a full-length native MCM. Variants do not encompass the native nucleotide sequence.

Ordinarily, MCM variant polynucleotides are at least about 30 nucleotides in length, often at least about 60, 90, 120, 150, 180, 210, 240, 270, 300, 450, 600 nucleotides in length, more often at least about 900 nucleotides in length, or more.

“Percent (%) nucleic acid sequence identity” with respect to MCM-encoding nucleic acid sequences identified herein is defined as the percentage of nucleotides in a candidate sequence that are identical with the nucleotides in the MCM sequence of interest, after algning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:
% nucleic acid sequence identity=W/Z·100

where

W is the number of nucleotides cored as identical matches by the sequence alignment program's or algorithm's alignment of C and D

and

Z is the total number of nucleotides in D.

When the length of nucleic acid sequence C is not equal to the length of nucleic acid sequence D, the % nucleic acid sequence identity of C to D will not equal the % nucleic acid sequence identity of D to C.

Homologs (i.e., nucleic acids encoding MCM derived from species other than human) or other related sequences (e.g., paralogs) can be obtained by low, moderate or high stringency hybridization with all or a portion of the particular human sequence as a probe using methods well known in the art for nucleic acid hybridization and cloning.

The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide which decreases DNA duplex stability. In general, the longer the probe, the higher the temperature required for proper annealing. A common approach is to vary the temperature: higher relative temperatures result in more stringent reaction conditions. (Ausubel et al., 1987) provide an excellent explanation of stringency of hybridization reactions.

To hybridize under “stringent conditions” describes hybridization protocols in which nucleotide sequences at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

In addition to naturally-occurring allelic variants of MCM, changes can be introduced by mutation into SEQ ID NO:8 that incur alterations in the amino acid sequences of the encoded MCM that do not alter MCM function. For example, nucleotide substitutions leading to amino acid substitutions at “non-essential” amino acid residues can be made in the sequence of SEQ ID NOs:9 and 10. A “non-essential” amino acid residue is a residue that can be altered from the wild-type sequences of the MCM without altering their biological activity, whereas an “essential” amino acid residue is required for such biological activity. For example, amino acid residues that are conserved among the MCM of the invention are predicted to be particularly non-amenable to alteration. Amino acids for which conservative substitutions can be made are well known in the art. Useful conservative substitutions are shown in Table 4, “Preferred substitutions.” Conservative substitutions whereby an amino acid of one class is replaced with another amino acid of the same type fall within the scope of the subject invention so long as the substitution does not materially alter the biological activity of the compound. If such substitutions result in a change in biological activity, then more substantial changes, indicated in Table 5 as exemplary are introduced and the products screened for MCM polypeptide biological activity.

TABLE 4
Preferred substitutions

| Original | | Preferred |
|---|---|---|
| residue | Exemplary substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, Norleucine | Leu |

Non-conservative substitutions that affect (1) the structure of the polypeptide backbone, such as a β-sheet or α-helical conformation, (2) the charge or (3) hydrophobicity, or (4) the bulk of the side chain of the target site can modify MCM polypeptide function or immunological identity. Residues are divided into groups based on common side-chain properties as denoted in Table 5. Non-conservative substitutions entail exchanging a member of one of these classes for another class. Substitutions may be introduced into conservative substitution sites or more preferably into non-conserved sites.

TABLE 5
Amino acid classes

| Class | Amino acids |
|---|---|
| hydrophobic | Norleucine, Met, Ala, Val, Leu, Ile |
| neutral hydrophilic | Cys, Ser, Thr |
| acidic | Asp, Glu |
| basic | Asn, Gln, His, Lys, Arg |
| disrupt chain conformation | Gly, Pro |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis (Carter, 1986; Zoller and Smith, 1987), cassette mutagenesis, restriction selection mutagenesis (Wells et al., 1985) or other known techniques can be performed on the cloned DNA to produce the MCM variant DNA (Ausubel et al., 1987; Sambrook et al., 1989).

In one embodiment, the isolated nucleic acid molecule comprises a nucleotide sequence encoding a protein, wherein the polypeptide comprises an amino acid sequence at least about 45%, preferably 60%, more preferably 64%, 65%, 66%, 67%, 68%, 69%, 70%, 80%, 90%, and most preferably about 95% homologous to SEQ ID NOs:9 and 10.

In general, a MCM variant that preserves MCM-like function and includes any variant in which residues at a particular position in the sequence have been substituted by other amino acids, and further includes the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

“MCM polypeptide variant” means an active MCM polypeptide having at least: (1) about 60%, more preferably 64%, amino acid sequence identity, with a full-length native sequence MCM polypeptide sequence, (2) a MCM polypeptide sequence lacking the signal peptide, (3) an extracellular domain of a MCM polypeptide, with or without the signal peptide, or (4) any other fragment of a full-length MCM polypeptide sequence. For example, MCM polypeptide variants include MCM polypeptides wherein one or more amino acid residues are added or deleted at the N— or C-terminus of the full-length native amino acid sequence. A MCM polypeptide variant will have at least about 60% amino acid sequence identity, preferably at least about 81 amino acid sequence identity, more preferably at least about 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% amino acid sequence identity and most preferably at least about 99% amino acid sequence identity with a full-length native sequence MCM polypeptide sequence. A MCM polypeptide variant may have a sequence lacking the signal peptide, an extracellular domain of a MCM polypeptide, with or without the signal peptide, or any other fragment of a full-length MCM polypeptide sequence. Ordinarily, MCM variant polypeptides are at least about 10 amino acids in length, often at least about 20 amino acids in length, more often at least about 30, 40, 50, 60, 70, 80, 90, 100, 150, 200, or 300 amino acids in length, or more.

“Percent (%) amino acid sequence identity” is defined as the percentage of amino acid residues that are identical with amino acid residues in the disclosed MCM polypeptide sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:
% amino acid sequence identity=X/Y·100

where

X is the number of amino acid residues scored as identical matches by the sequence alignment program's or algorithm's alignment of A and B

and

Y is the total number of amino acid residues in B.

If the length of amino acid sequence A is not equal to the length of amino acid sequence B, the % amino acid sequence identity of A to B will not equal the % amino acid sequence identity of B to A.

Biologically active portions of MCM include peptides comprising amino acid sequences sufficiently homologous to or derived from the amino acid sequences of the MCM (SEQ ID NOs:9 and 10) that include fewer amino acids than the full-length MCM, and exhibit at least one activity of a MCM. Biologically active portions comprise a domain or motif with at least one activity of native MCM. A biologically active portion of a MCM can be a polypeptide that is, for example, 10, 25, 50, 100 or more amino acid residues in length. Other biologically active portions, in which other regions of the protein are deleted, can be prepared by recombinant techniques and evaluated for one or more of the functional activities of a native MCM.

Biologically active portions of MCM may have an amino acid sequence shown in SEQ ID NOs:9 and 10, or substantially homologous to SEQ ID NOs:9 and 10, and retains the functional activity of the protein of SEQ ID NOs:9 and 10, yet differs in amino acid sequence due to natural allelic variation or mutagenesis. Other biologically active MCM may comprise an amino acid sequence at least 45% homologous to the amino acid sequence of SEQ ID NOs:9 and 10, and retains the functional activity of native MCM.

Vectors act as tools to shuttle DNA between host cells or as a means to produce a large quantity of the DNA. Some vectors function only in prokaryotes, while others function in both prokaryotes and eukaryotes, enabling large-scale DNA preparation from prokaryotes to expression in a eukaryote. Inserting the DNA of interest, such as MCM nucleotide sequence or a fragment, is accomplished by ligation techniques and/or transformation protocols well-known to the skilled artisan. Such DNA is inserted such that its integration does not disrupt any necessary components of the vector. In the case of vectors that are used to express the inserted DNA protein, the introduced DNA is operably linked to the vector elements that govern its transcription and translation.

Vectors often have a selectable marker that facilitates identifying those cells that have taken up the exogenous nucleic acids. Many selectable markers are well known in the art for the use with prokaryotes, usually antibiotic-resistance genes or the use of autotrophy and auxotrophy.

Vector choice is governed by the organism or cells being used and the desired fate of the vector. Vectors replicate once in the target cells or can be “suicide” vectors. In general, vectors comprise signal sequences, origins of replication, marker genes, enhancer elements, promoters, and transcription termination sequences. The choice of these elements depends on the organisms in which they are used and are easily determined by one of skill in the art. Some of these elements may be conditional, such as an inducible or conditional promoter that is turned “on” when conditions are appropriate. Examples of such promoters include tissue-specific, which relegate expression to certain cell types, steroid-responsive, heat-shock inducible, and prokaryotic promoters.

Methods of eukaryotic cell transfection and prokaryotic cell transformation are well known in the art and can be used to recombinantly produce MCM protein. The choice of host cell dictates the preferred technique for introducing the nucleic acid of interest. Introduction of nucleic acids into an organism can also be done with ex vivo techniques that use an in vitro method of transfection.

To monitor MCM gene expression or to facilitate biochemical purification, MCM nucleotide sequence can be fused to a heterologous peptide. These include reporter enzymes and epitope tags that are bound by specific antibodies.

(c) Increasing Translation

Any method known in the art to increase translation of MCM polynucleotides can be used. These include providing extra energy (e.g., sugars, starches, adenosine tri-phosphate (ATP) and the like) to the media, translation building blocks, such as purified, or partially purified amino acids or derivatives thereof, or even altering the temperature of the culture.

Proper expression in a prokaryotic cell also requires the presence of a ribosome binding site upstream of the gene sequence-encoding sequence. Such ribosome binding sites are known in the art, (see, e.g., (Gold et al., 1981)). The ribosome binding site and other sequences required for translation initiation are operably linked to the nucleic acid molecule coding for MCM by, for example, in frame ligation of synthetic oligonucleotides that contain such control sequences. The selection of control sequences, expression vectors, transformation methods, and the like, are dependent on the type of host cell used to express the gene.

(d) Other

Compounds that are amplifiers, transcription up-regulators, translation up-regulators or agonists, are effective to increase MCM activity Conversely, compounds that are de-amplifiers, transcription down-regulators, translation down-regulators or antagonists, are effective to increase MCM activity when these compounds act on negative regulators of MCM activity.

Decreasing Negative Regulator Activity

The transcription of negative regulators can be inhibited using means well known in the art. For example, DNA binding proteins such as zinc fingers are known to bind to and inhibit transcription of genes (see, e.g., (Barbas et al., 2000)). A preferred means for inhibiting negative regulator activity is to mutate the wild-type gene to express a reduced-activity mutant form, or to not express any gene at all. Promoter sequences operably linked to the regulator gene are also preferred targets to reduce or eliminate expression. Means for mutating genes are well known in the art; e.g. see (Ausubel et al., 1987; Sambrook et al., 1989).

Using antisense and sense MCM oligonucleotides can prevent MCM polypeptide expression. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are singe-stranded nucleic acids, either RNA or DNA, which can bind target MCM mRNA (sense) or MCM DNA (antisense) sequences and inhibit transcription, translation, or both of MCM. Anti-sense nucleic acids can be designed according to Watson and Crick or Hoogsteen base pairing rules. The anti-sense nucleic acid molecule can be complementary to the entire coding region of MCM mRNA, but more preferably, to only a portion of the coding or noncoding region of MCM mRNA. For example, the anti-sense oligonucleotide can be complementary to the region surrounding the translation start site of MCM mRNA. Antisense or sense oligonucleotides may comprise a fragment of the MCM DNA coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Among others, (Stein and Cohen, 1988; van der Krol et al., 1988a) describe methods to derive antisense or a sense oligonucleotides from a given cDNA sequence.

Examples of modified nucleotides that can be used to generate the anti-sense nucleic acid include: 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the anti-sense nucleic acid can be produced biologically using an expression vector into which a nucleic acid has been sub-cloned in an anti-sense orientation such that the transcribed RNA will be complementary to a target nucleic acid of interest.

To introduce antisense or sense oligonucleotides into target cells (cells containing the target nucleic acid sequence), any gene transfer method may be used. Examples of gene transfer methods include (1) biological, such as gene transfer vectors like Epstein-Barr virus, conjugating the exogenous DNA to a ligand-binding molecule, or by mating, (2) physical, such as electroporation and injection, and (3) chemical, such as CaPO₄ precipitation and oligonucleotide-lipid complexes.

An antisense or sense oligonucleotide is inserted into a suitable gene transfer retroviral vector. A cell containing the target nucleic acid sequence is contacted with the recombinant retroviral vector, either in vivo or ex vivo. For eukaryotes, examples of suitable retroviral vectors include those derived from the murine retrovirus M-MuLV, N2 (a retrovirus derived from M-MuLV), or the double copy vectors designated DCT5A, DCT5B and DCT5C (1990b). For prokaryotes, a plethora of vectors are available, including those disclosed in the Examples (below), and classic plasmids including pBR322. Transposons can also be used. To achieve sufficient nucleic acid molecule transcription, vector constructs in which the transcription of the anti-sense nucleic acid molecule is controlled by a strong and/or inducible promoter are preferred.

A useful anti-sense nucleic acid molecule can be an α-anomeric nucleic acid molecule. An α-anomeric nucleic acid molecule forms specific double-stranded hybrids with complementary RNA in which, contrary to the usual α-units, the strands run parallel to each other (Gautier et al., 1987). The anti-sense nucleic acid molecule can also comprise a 2′-o-methylribonucleotide (Inoue et al., 1987a) or a chimeric RNA-DNA analogue (Inoue et al., 1987b).

In one embodiment, an anti-sense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes, such as hammerhead ribozymes (Haseloff and Gerlach, 1988) can be used to catalytically cleave MCM mRNA transcripts and thus inhibit translation. A ribozyme specific for a MCM-encoding nucleic acid can be designed based on the nucleotide sequence of a MCM cDNA (i.e., SEQ ID NO:8). For example, a derivative of a Tetrahymena a L-19 IVS RNA can be constructed in which the nucleotide sequence of the active site is complementary to the nucleotide sequence to be cleaved in a MCM-encoding mRNA (Cech et al., 1992; Cech et al., 1991). MCM mRNA can also be used to select a catalytic RNA having a specific ribonuclease activity from a pool of RNA molecules (Bartel and Szostak, 1993).

Alternatively, MCM expression can be inhibited by targeting nucleotide sequences complementary to the regulatory region of the MCM (e.g., the MCM promoter and/or enhancers) to form triple helical structures that prevent transcription of the MCM in target cells (Helene, 1991; Helene et al., 1992; Maher, 1992).

Modifications of antisense and sense oligonucleotides can augment their effectiveness. Modified sugar-phosphodiester bonds or other sugar linkages (1991), increase in vivo stability by conferring resistance to endogenous nucleases without disrupting binding specificity to target sequences. Other modifications can increase the affinities of the oligonucleotides for their targets, such as covalently linked organic moieties (1990a) or poly-(L)-lysine. Other attachments modify binding specificities of the oligonucleotides for their targets, including metal complexes or intercalating (e.g. ellipticine) and alkylating agents.

For example, the deoxyribose phosphate backbone of the nucleic acids can be modified to generate peptide nucleic acids (Hyrup and Nielsen, 1996). “Peptide nucleic acids” or “PNAs” refer to nucleic acid mimics (e.g., DNA mimics) in that the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols (Hyrup and Nielsen, 1996; Perry-O'Keefe et al., 1996).

PNAs of MCM can be used in therapeutic and diagnostic applications. For example, PNAs can be used as anti-sense or antigene agents for sequence-specific modulation of gene expression by inducing transcription or translation arrest or inhibiting replication. MCM PNAs may also be used in the analysis of single base pair mutations (e.g., PNA directed PCR clamping; as artificial restriction enzymes when used in combination with other enzymes, e.g., S₁ nucleases (Hyrup and Nielsen, 1996); or as probes or primers for DNA sequence and hybridization (Hyrup and Nielsen, 1996; Perry-O'Keefe et al., 1996).

PNAs of MCM can be modified to enhance their stability or cellular uptake. Lipophilic or other helper groups may be attached to PNAs, PNA-DNA dimmers formed, or the use of liposomes or other drug delivery techniques. For example, PNA-DNA chimeras can be generated that may combine the advantageous properties of PNA and DNA. Such chimeras allow DNA recognition enzymes (e.g., RNase H and DNA polymerases) to interact with the DNA portion while the PNA portion provides high binding affinity and specificity. PNA-DNA chimeras can be linked using linkers of appropriate lengths selected in terms of base stacking, number of bonds between the nucleobases, and orientation (Hyrup and Nielsen, 1996). The synthesis of PNA-DNA chimeras can be performed (Finn et al., 1996; Hyrup and Nielsen, 1996). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Finn et al., 1996; Hyrup and Nielsen, 1996). PNA monomers are then coupled in a stepwise manner to produce a chimeric molecule with a 5′ PNA segment and a 3′ DNA segment (Finn et al., 1996). Alternatively, chimeric molecules can be synthesized with a 5′ DNA segment and a 3′ PNA segment (Petersen et al., 1976).

The oligonucleotide may include other appended groups such as peptides (e.g., for targeting host cell receptors in vivo), or agents facilitating transport across the cell membrane (Lemaitre et al., 1987; Letsinger et al., 1989) or PCT Publication No. WO88/09810) or the blood-brain barrier (e.g., PCT Publication No. WO 89/10134). In addition, oligonucleotides can be modified with hybridization-triggered cleavage agents (van der Krol et al., 1988b) or intercalating agents (Zon, 1988). The oligonucleotide may be conjugated to another molecule, e.g., a peptide, a hybridization triggered cross-linking agent, a transport agent, a hybridization-triggered cleavage agent, and the like.

Cells

A cell can be a prokaryotic or eukaryotic cell. A preferred prokaryotic cell is a bacterial cell. Preferred and exemplary bacterial cells are Saccharopolyspora, Aeromicrobium and Streptomyces. Particularly preferred bacterial cells are Saccharopolyspora erythraea, Aeromicrobium erythreum, Streptomyces fradiae, Streptomyces avermitilis, Streptomyces cinnamonensis, Streptomyces antibioticus, Streptomyces venezuelae, Streptomyces violaceoniger, Streptomyces hygroscopicus, Streptomyces spp. FR-008, and Streptomyces griseus. These an other bacterial strains are available from American Type Tissue Collection (ATCC); Manassus, Va.) and Northern Regional Research Laboratory (Peoria, Ill.). Examples of just some, not all, useful strains are shown in Table 6.

Any eukaryotic cell can be used, although mammalian cells are preferred. Primary culture cells, as well as cell lines (available from the ATCC are useful, although cell lines are preferred because of their immortality and ease of manipulation.

TABLE 6
Examples of useful strains

| | ATCC/NRRL | |
|---|---|---|
| Strain | Deposit | Notes |
| S. erythreae | ATCC 11912 | Originally deposited as |
| | | Streptomyces erythraeus; |
| | | Designation: 3036 [PSA 43] |
| S. erythreae | ATCC 31772 | Originally deposited as |
| | | Streptomyces erythraeus; |
| | | Designation: LMC 1648 |
| S. erythreae | ATCC 55441 | |
| S. erythreae | ATCC 11635 | Originally deposited as |
| | | Streptomyces erythraeus; |
| | | Designation: M5-12259 |
| A. erythreum | ATCC 51598 | Designation: NRRL B-3381 |
| S. fradiae | ATCC 11903 | Designation IFO 3123 |
| S. fradiae | ATCC 31669 | Designation: A252.7 |
| S. fradiae | ATCC 15861 | Designation: RIA 571 |
| S. fradiae | ATCC 21696 | Designation: K162 |
| S. fradiae | ATCC 10147 | Designation: 3034 |
| S. fradiae | ATCC 10745/NRRL | Designation: 3535 |
| | B-1195 | |
| S. fradiae | ATCC 14443 | Designation: Chas. |
| | | Pfizer Co. FD 44490-1 |
| S. fradiae | ATCC 14544 | Designation: IMRU 3739 |
| S. fradiae | ATCC 15438 | Designation: 3556A |
| S. fradiae | ATCC 19063 | Designation: KY 631 |
| S. fradiae | ATCC 19609/NRRL | Designation: M48-E2724 |
| | B-2702 | |
| S. fradiae | ATCC 19760 | Designation: ISP 5063 |
| S. fradiae | ATCC 19922 | Designation: INA 14250 |
| S. fradiae | ATCC 21097/NRRL | Designation: MA-2911 |
| | B-3358 | |
| S. fradiae | ATCC 21099/NRRL | Designation: MA-2913 |
| | B-3360 | |
| S. fradiae | ATCC 21096/NRRL | Designation: MA-2898 |
| | B-3357 | |
| S. fradiae | ATCC 21098/NRRL | Designation: MA-2912 |
| | B-3359 | |
| S. fradiae | ATCC 21896 | Designation: IFO 3360 |
| S. fradiae | ATCC 31846 | Designation: YO-9010 |

Suitable media and conditions for growing the modified bacteria include using SCM and Insoluble Production Medium (IPM; typically 22 g soy flour, 15 g corn starch, 3 g CaCO₃, 0.5 g MgSO₄.7H₂O and 15 mg FeSO₄.7H₂O/liter). However, any media which supports the increased activity of MCM can be used. A key factor, however, is the use of an unrefined soy source, such as soy flour. Media that are used industrially are especially preferred. Numerous formulations are known in the art; e.g., see (Ausubel et al., 1987).

An important aspect of the present invention is the presence or absence of soybean oil. In most instances, the use of soybean oil is preferred. However, when used, the concentration (v/v) is about 1% to 10%, preferably 2.5% to 7%, more preferably 4% to 6%, and most preferably 5%. If oil is omitted from the medium, then starch content is preferably increased. Typically, a 1.5- to 10-fold increase, preferably a 2- to 7-fold, more preferably 3- to 5-fold, and most preferably, a 4-fold increase.

Another aspect of the invention includes embodiments wherein the cultures are agitated more than typically. Agitation, in any case, is desired to increase culture aeration. In shaker flasks cultures, agitations can be 100 rpm to 1000; preferably 200 to 750 rpm, more preferably 350 to 500 rpm, and most preferably 400 rpm; in these examples, displacement used for shaking is approximately one inch. The mode of agitation can vary; those of skill in the art can translate these agitation conditions to the vessels and methods of agitation for their particular situation.

Temperature is also regulated; typically for S. erythraea, a temperature of 32° C. is preferred. Humidity is also regulated; for example, incubator humidity controls can be set to 50% to 100%, preferably 60% to 80%, and most preferably 65%.

### EXAMPLES

The following example is for illustrative purposes only and should not be interpreted as limitations of the claimed invention. There are a variety of alternative techniques and procedures available to those of skill in the art which would similarly permit one to successfully perform the intended invention.

### Example 1

### Methods and Materials—MCM mutants in an Industrial Erythromycin-Producing Strain and Erythromycin Production

Bacterial Strains and Culture Conditions

The bacterial strains and plasmids used in this study are shown in Table 7. Saccharopolyspora erythraea ATCC 11635. S. erythraea FL2267 is a derivative of ATCC 11635, an industrial erythromycin-producing strain, that was generated by eviction of an integrated plasmid and reversion to the wild-type thiostrepton-sensitive phenotype. FL1347 is a low erythromycin-producing red variant of ATCC 11635 generated at Fermalogic, Inc. (Chicago, Ill.) by spontaneous mutation. The white wild-type strain and derivatives were cultured on E20A agar plates (E20A per liter tap water: 5 g, bacto soytone; 5 g, bacto soluble starch; 3 g, CaCO₃, 2.1 g 3-(N-Morpholino)propanesulfonic acid (MOPS); 20 g, Difco agar (Becton-Dickinson; Franklin Lakes, N.J.); after autoclaving added 1 ml of thiamine (1.0% solution) and 1 ml of FeSO₄ (1.2% solution)) or R2T2 agar (Weber et al., 1990). Red variants were cultured on R2T2 agar. For liquid culture cells were grown in Soluble Complete Medium (SCM) pH 6.8, (McAlpine et al., 1987); SCM per liter: 15 g soluble starch; 20 g bacto soytone (soybean peptone; Becton-Dickinson); 0.1 g calcium chloride; 1.5 g yeast extract; 10.5 g MOPS). For experiments with minimal media AVMM was used (Weber and McAlpine, 1992). Sole carbon sources, such as methylmalonic acid, sucrose and glucose were added to a final concentration of 50 mM. Ammonium sulfate was used as the sole nitrogen source at a final concentration of 7.5 mM. Escherichia coli DH5α-e (Invitrogen; Carlsbad, Calif.) was routinely grown in SOB or 2×YT liquid media and maintained on SOB or 2×YT agar (Sambrook et al., 1989). For agar plate bioassays the thiostrepton-resistant Bacillus subtilis PY79 was used as the indicator strain (Weber et al., 1990). When appropriate for growth of drug-resistant S. erythraea, solid and liquid media were supplemented with either thiostrepton at a final concentration of 10 μg/ml or kanamycin sulfate at a final concentration of 50 μg/ml (Sigma-Aldrich; St. Louis, Mo.). E. coli media were supplemented with 50 μg/ml kanamycin sulfate or 100 μg/ml ampicillin sodium salt (Sigma-Aldrich) for selection and maintenance of recombinant plasmids.

TABLE 7
Bacterial strains and plasmids used in this study

| Plasmid | | Reference |
|---|---|---|
| or strain | Description | or source |
| pFL8 | S. erythraea suicide vector. Used to make | (Reeves et |
| | gene knockouts in the chromosome. Thio^{r}. | al., 2002) |
| pARR11 | S. erythraea integration vector | (Weber and |
| | containing a 5.68 kb EcoRI, HindIII | Losick, |
| | fragment from pMW3. Thio^{r}. | 1988) |
| pFL2107 | Plasmid used to make a knockout by | This study |
| | single crossover insertion of an | |
| | internal mutB fragment. Contains a | |
| | 1.32 kb fragment cloned into pFL8. Thio^{r}. | |
| pFL2114 | PGEM ® T Easy (Promega; Madison, WI) | This study |
| | containing a 742 bp region internal to | |
| | meaB. Used for subcloning into pFL8. Ap^{r}. | |
| pFL2132 | S. erythraea integration vector used to | This study |
| | make a knockout of mutB by gene | |
| | replacement and insertion of a kanamycin | |
| | resistance gene cassette. Contains two | |
| | non-contiguous fragments from the mutAB | |
| | region. Thio^{r}, Kn^{r}. | |
| pFL2179 | Derivative of pFL2132 that has lost the | This study |
| | kanamycin resistance gene cassette by | |
| | BamHI digestion followed by religation. | |
| | Used to make in-frame deletion in mutB. | |
| | Thio^{r}, Kn^{s}. | |
| pFL2121 | S. erythraea integration vector used to | This study |
| | make a knockout of meaB by single | |
| | crossover insertion of a 742 bp internal | |
| | fragment. Thio^{r} | |
| pFL2212 | S. erythraea integration vector used | This study |
| | to insert a duplicate copy of the | |
| | methylmalonyl-CoA mutase region in the | |
| | chromosome. The total region integrated | |
| | was 6.791 kb and contained the entire | |
| | SeORF1, mutA, mutB, meaB, and gntR | |
| | genes (DNA accession nos. DQ289499 and | |
| | DQ289500). | |
| FL2267 | Derivative of S. erythraea ATCC 11635. | This study |
| | Wild-type revertant obtained by eviction | |
| | of an integrated plasmid. Used as host | |
| | strain in transformations. | |
| FL1347 | Red variant of S. erythraea ATCC 11635. | Reeves |
| | Low erythromycin producer. Used as host | et al., |
| | strain in transformations. | (2002). |
| FL2272 | Derivative of FL2267 containing | This study |
| | integrated pFL2132 by single crossover | |
| | insertion. Thio^{r}, Kn^{r}. | |
| FL2155 | Derivative of FL1347 containing | This study |
| | integrated pFL2107 by single crossover | |
| | insertion. Thio^{r}, Kn^{s}. | |
| FL2294 | Derivative of FL2267 containing | This study |
| | integrated pFL2179 by single crossover | |
| | insertion. Thio^{r}, Kn^{s}. | |
| FL2281 | Gene replacement derivative of FL2272 | This study |
| | obtained by eviction of pFL2132. | |
| | Thio^{s} Kn^{r}. | |
| FL2302 | Gene replacement derivative of FL2294 | This study |
| | obtained by eviction of pFL2179. Kn^{s}, | |
| | Thio^{s}. | |
| FL2320 | Derivative of FL2267 containing | This study |
| | integrated pFL2121 by single crossover | |
| | insertion. Thio^{t} | |
| FL2385 | Derivative of FL2267 containing | This study |
| | integrated pFL2212 by single crossover | |
| | insertion. Thio^{r}. | |
| DHSα | E. coli host strain for transformations | Invitrogen |
| | | (Carlsbad, |
| | | CA) |

Plasmid Constructions

pFL2132, polar knockout plasmid To generate a knockout in mutB, a polymerase chain reaction (PCR) approach was used. Primers were designed so that two non-contiguous fragments spanning the mutAB gene region were amplified. Primer pair A, 5′-gaattcCCGTGCGCCCGTTCGACGC-3′ (SEQ ID NO:1) and 5′-ggatccGTGTTGCGGGCGATGCGCG-3′ (SEQ ID NO:2; lowercase letters indicate engineered sequences containing restriction sites), generated a 1997 base-pair (bp) product that spanned from mutA to the middle of mutB (Reeves et al., 2004). Primer pair B, aagcttAGCGTGTCCAGGCCCGCTC-3′ (SEQ ID NO:3) and 5′-ggatccGACGCAGGCGCGCATCGACT-3′ (SEQ ID NO:4; lowercase letters indicate engineered sequences containing restriction sites) generated a 1666 bp product that spanned from mutB to near the end of meaB (Reeves et al., 2004). The region of discontiguity was 126 bp, located near the middle of mutB. Restriction sites were engineered at the 5′ ends of each primer pair to facilitate later cloning steps. Both PCR products were cloned directly into pGEM® T easy.

To generate the knockout plasmid pFL2132, a four-component ligation reaction was performed. This consisted of pFL8 digested with EcoRI and HindIII (Reeves et al., 2002), the kanamycin resistance gene cassette from Tn903 (Pharmacia Biochemicals; Piscataway, N.J.) digested with BamHI and the two PCR products released from pGEM® T easy. An EcoRI+BamHI digest was used in the case of the 1997 bp fragment and a BamHI+HindIII digest in the case of the 1666 bp fragment. E. coli was transformed by electroporation and recombinants were selected for kanamycin and ampicillin resistance. Plasmids were confirmed for the correct inserts by restriction digestion and sequence analysis.

pFL2179, in-frame deletion plasmid To generate an in-frame mutB deletion mutant, pFL2132 was digested with BamHI to release a unique 1263 bp fragment consisting entirely of the kanamycin resistance gene cassette. The remaining larger fragment was purified from an agarose gel and re-ligated using T4 DNA ligase (Fermentas; Vilnius, Lithuania). The truncated plasmid was transformed into E. coli. Single ampicillin-resistant colonies were replica patched onto SOB agar containing kanamycin and ampicillin. Isolates that were ampicillin-resistant but kanamycin-sensitive were further analyzed. Ten plasmids from kanamycin-sensitive isolates were digested with BamHI and HindIII to confirm the loss of the kanamycin resistance gene cassette. This plasmid contains a 126 bp deletion in mutB along with an engineered BamHI site (6 bp) to maintain the reading frame of the gene.

pFL2121, meaB knockout plasmid Construction of a meaB knockout plasmid was performed using a PCR approach. Oligonucleotide primers were designed to amplify a 742 bp internal region of meaB. The primer sequences were as follows (lowercase letters indicate engineered sequences containing restriction sites): 5′-gtcgaattcAGCACCGCGCGAAAGCCCAG-3′ (SEQ ID NO:5) and 5′-gtcaagcttTAAGCTGGAGCAGCTGCTAC-3′ (SEQ ID NO:6). Following purification, the PCR product was cloned directly into pGEM® T easy as described above. The meaB fragment, released by EcoRI and HindIII digestion, was sub-cloned into the S. erythraea integration vector pFL8 (Reeves et al., 2002), which had been previously digested with the same enzymes. This plasmid was designated pFL2121 (Table 7). Transformation of pFL2121 DNA into S. erythraea strain FL2267 was performed as described below. The S. erythraea FL2267 containing integrated pFL2121 was designated FL2320 (Table 7). pFL2212 plasmid was used to duplicate the methylmalonyl-CoA region in the S. erythraea chromosome. The entire S. erythraea methylmalonyl-CoA mutase operon was cloned from a cosmid as a 6.791 kb EcoRI/BamHI fragment into pFL8 cut with the same enzymes (Reeves et al., 2002). The cloned fragment was confirmed by sequence analysis and restriction digestion. The plasmid DNA was introduced into S. erythraea wild-type strain FL2267 by protoplast transformation with selection for thiostrepton resistance. Spores of putative thiostrepton-resistant transformants from separate transformations were tested in a second round of thiostrepton selection by plating on E20A agar plates and growing in SCM broth containing thiostrepton at a final concentration of 15 μg/ml. Chromosomal DNA was prepared from five different isolates for PCR analysis to confirm the integration of the plasmid. All five isolates gave the expected PCR product. The S. erythraea strains containing a duplicate copy of the mmCoA mutase operon was designated FL2385.

Generation of mutB mutants Five types of mutB mutants were generated in this study. These consisted of the three, single crossover mutants generated by integration of pFL2107, pFL2132 and pFL2179, and the double crossover (gene replacement) mutants generated by eviction of pFL2132 and pFL2179 with retention in the chromosome of the mutated copy of mutB. All subsequent results described below for the white strain derivatives were obtained from strains derived by gene replacement of the mutated copy of mutB. These mutants were advantageous for several reasons, the main ones being: (i) the permanence or stability of the mutation during growth; and (ii) isolation of the mutation to only the mutB reading frame in the case of S. erythraea strain FL2302. Analysis of the white strain single crossover mutants was taken into account but was not involved in the final interpretation of the results since these types of mutations do not necessarily knock out a gene. Results obtained in the red strain were from a single crossover knockout strain generated by integration of pFL2107 (FL2155; Table 7). Transformations of pFL2132 and pFL2179 were performed with selection for thiostrepton resistance. These transformations generated the single crossover mutants FL2272 and FL2294, respectively. After confirmation of plasmid integration, cells were subjected to a plasmid eviction procedure to generate both double crossover (gene replacement) mutants as well as wild type revertant strains. The gene replacement strains containing the kanamycin resistance gene cassette inserted into mutB was designated FL2281 and the in-frame deletion strain was designated FL2302.

Transformations Protoplast transformation of the S. erythraea wild type (white) strain is known to be difficult to perform successfully, in contrast to red variant strains. To increase the likelihood of transforming the S. erythraea wild-type strain a new host strain was generated. The ATCC 11635 derivative, FL2267, a wild type revertant, was used in all transformations. This strain was generated from eviction of integrated pARR11, a S. erythraea vector inserted into the chromosome by single crossover integration of homologous DNA (Table 7; (Reeves et al., 2002; Weber and Losick, 1988)). Putative evictants were streaked for single colonies onto E20A agar plates and allowed to sporulate. Individual colonies were replica patched onto fresh E20A agar plates containing thiostrepton at 10 μg/ml or no antibiotic to test for loss of the plasmid. Isolates that were confirmed to be thiostrepton sensitive were later used as hosts in protoplast transformations. Protoplast transformations using pFL2132 and pFL2179 DNA (10 μg total) were performed as described (Reeves et al., 2002), using either thiostrepton (final concentration of 8 μg/ml) or kanamycin sulfate (final concentration of 10 μg/ml) as the selection agent.

Fermentations Shake flask fermentations were performed in SCM (“medium 1;” (McAlpine et al., 1987)), SCM +5% v/v soybean oil (medium 2), SCM+4× soluble starch (medium 3) and SCM+4× starch and 5% v/v soy oil (medium 4). Cultures were incubated at 32.5° C. for 5 days at 350 rpm to 425 rpm. The fermentations were performed on an INFORS minitron (ATR; Laurel, Md.) with humidity control. Humidity was set at 65% throughout the incubation period.

Bioassay for erythromycin production Bioassays for the determination of erythromycin production of shake flask cultures was performed as described (Reeves et al., 2002).

Phenotype testing S. erythraea mutB mutants were tested for various phenotypes on E20A agar and minimal medium AVMM agar (Weber and McAlpine, 1992; (Reeves et al., 2004)). Growth on methylmalonic acid as sole carbon source was tested on AVMM agar supplemented with 50 mM methylmalonic acid (Sigma-Aldrich, St. Louis, Mo.). Pigment production was tested on AVMM agar supplemented with 50 mM glucose and R2T2 agar. The ability to form aerial mycelia and to sporulate was tested on E20A agar.

Statistical analysis t-Tests and probability values were calculated for 95% confidence intervals using interactive software (Uitenbroek, 2005).

### Example 2

### Growth, Pigmentation and Sporulation Phenotypes of mutB Mutants. Red Variant Mutants

Previous results from S. erythraea red variant mutB mutants showed a pleiotropic effect of the mutation. In those strains, major phenotypic differences were observed in the mutants compared to the parent strain in their ability to: (i) produce diffusible red pigment; (ii) grow on methylmalonic acid as the sole carbon source; and (iii) form aerial mycelia followed by complete septation of spores.

The same experiments were performed with the white S. erythraea mutB mutant strains. Cells of FL2281 and FL2302, along with parent and single crossover strains as controls, were plated onto four different plates: (i) E20A (a rich medium) and three separate AVMM plates containing either (ii) glucose, (iii) methylmalonic acid, or (iv) glucose and succinate as sole carbon sources. As observed with the red variant mutB mutants, both types of white strain mutant exhibited the same pleiotropic effects of the mutation. Both FL2281 and FL2302 were unable to grow on methylmalonic acid as sole carbon source. The wild type strain and wild type revertant strains grew well, indicating fully functional mutase activity. A single crossover strain showed poor growth, indicating a decrease in mutase activity.

Diffusible red pigment production was lost in all the mutant strains. Pigment production was observed in the wild type strain and, importantly, it was restored in the wild type revertant strains.

Sporulation was also affected in both types of mutB mutants. In a simple test for spore formation, the wild type and mutB mutant strain were spread on half of the same E20A agar plate as a lawn and allowed to grow for 10 days at 33° C., more than enough time for complete sporulation. After incubation, the spores were scraped and transferred with a wooden stick to 1 ml of water. The wild type spores disbursed evenly and quickly without vortexing. The spores of the mutB mutant formed clumps on both the wooden stick and in liquid. No dispersal occurred even after vigorous vortexing for 1 minute.

### Example 3

### Erythromycin Production of mutB Muntants

In these experiments, the ability of the mutated strains to produce erythromycin was tested. Shake flask fermentations were performed on mutB mutants to first determine whether the mutation increased erythromycin production. The results of these experiments were used to optimize antibiotic production by implementing process improvements. Process improvements that were implemented once an increase in production was observed in mutB mutants were the addition of three-fold more soluble starch and the elimination of soybean oil. Shaker speed was increased from 350 rpm to 390 rpm.

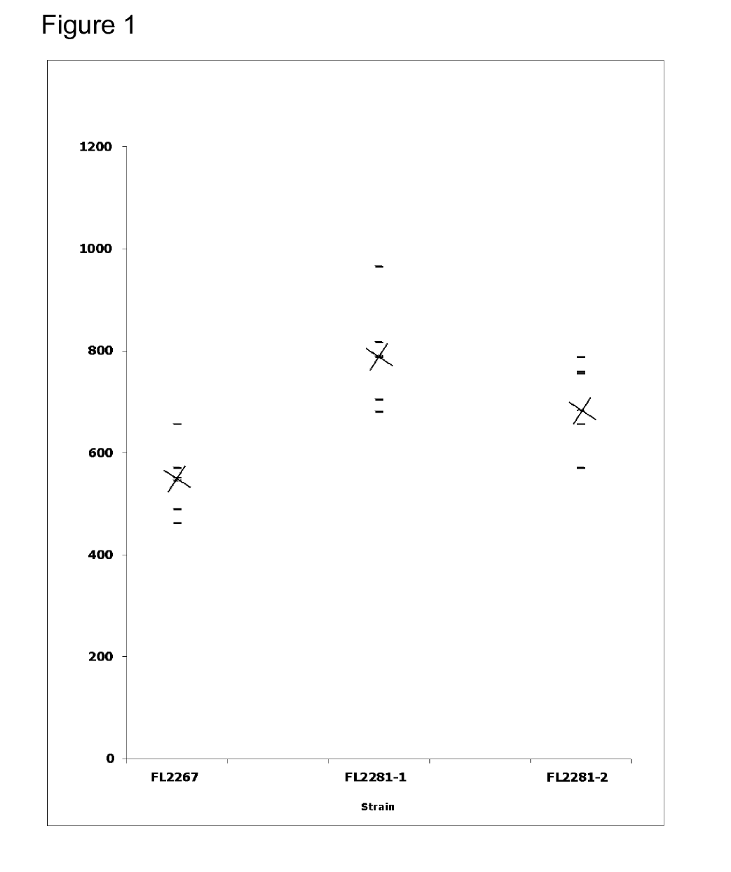

Initial fermentations consisted of shake flask cultures of S. erythraea wild type strain and mutB mutant in medium 2 (SCM+5% soybean oil). Cultures were incubated at 32.5° C. for 5 days at 350 rpm with humidity at a constant 65%. Shake flasks were inoculated with a 2-day seed culture at a 1:10 dilution, and the results are shown in FIG. 1. “X's” indicate the average erythromycin yield of triplicate fermentations and two replicate bioassay disks for each culture. As shown in FIG. 1, S. erythraea strain FL2281 produced on average 25% more erythromycin than the parent strain FL2267 when grown in medium 2.

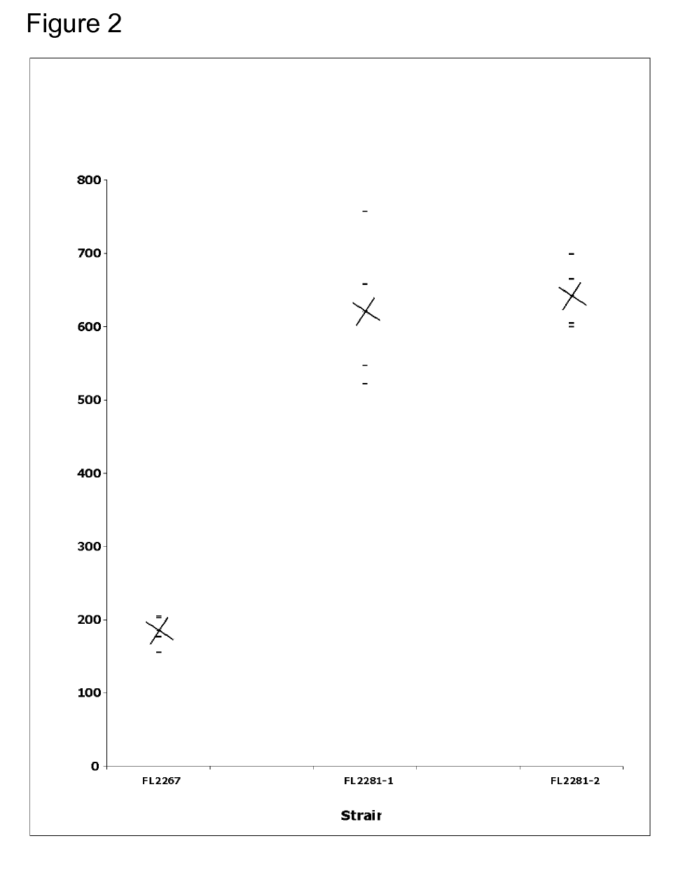

It was not known what effect omitting soybean oil in the medium would have on mutB strains since soybean oil has been suggested to be involved in both erythromycin precursor feeding and in increasing cell density (Li et al., 2004). However, when cells were grown in the absence of soybean oil (medium 1), the difference in erythromycin production between the parent strain and the mutB mutant was dramatic. The wild-type strain produced significantly less erythromycin (about 67%) in medium 1 when compared to the production of the strain cultured in medium 2, as shown in FIG. 2; “X's” indicate the production averages. Surprisingly, the mutB mutants produced the same amount of erythromycin in medium 1 as in medium 2. Overall, the mutB mutant made on average 2.5-fold more erythromycin than the parent strain in the absence of soybean oil.

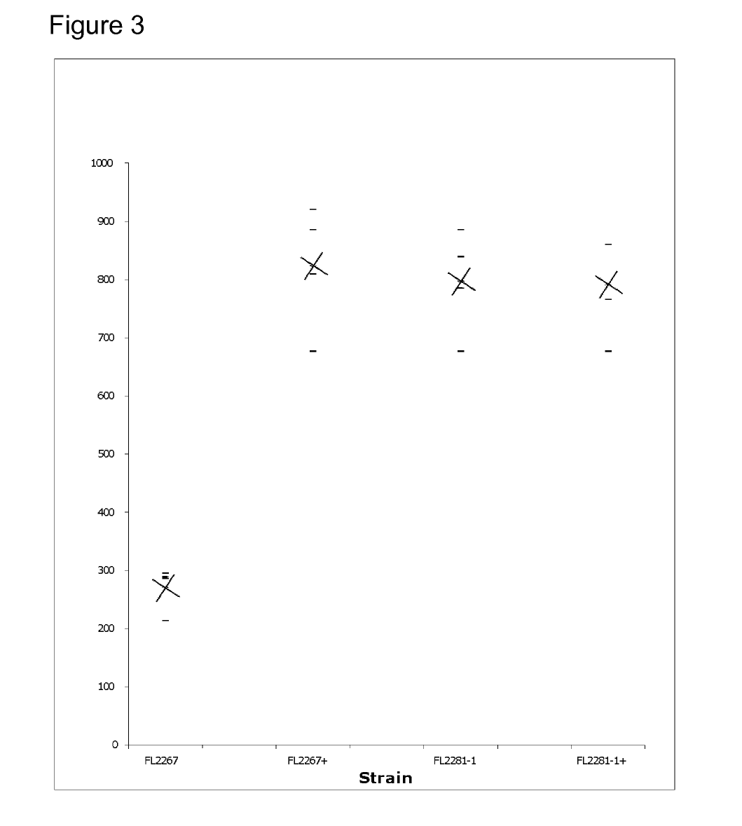

When the wild-type and mutB strains were grown in medium 1 and medium 2 during the same fermentation, the same trend in erythromycin production levels as again observed, as shown in FIG. 3; “X's” indicate the production averages. Wild-type S. erythraea produced erythromycin best in the presence of oil, whereas mutB mutants produce erythromycin at a similar level to the wild-type strain in either the presence or absence of soybean oil. Therefore, the presence of soybean oil had no noticeable effect on overall erythromycin production in mutB mutants.

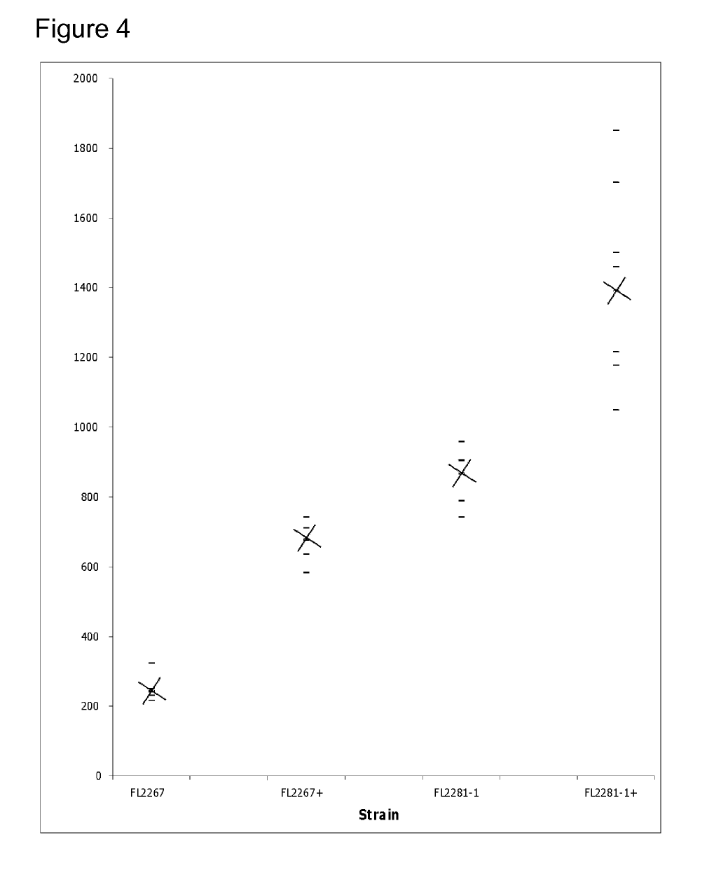

Since mutB mutants do not benefit from the addition of soybean oil, starch content of the medium was increased to provide additional carbon sources that are missing when soybean oil is omitted. The overall effect on erythromycin production, particularly in the mutB mutant, was dramatic, as shown in FIG. 4; “X's” indicate the average production. The wild type strain in medium 3 produced about as much erythromycin as when grown in medium 2 (˜600-700 μg/ml), the difference being the additional starch and lack of oil in medium 3. Strikingly, mutB mutants produced significantly more erythromycin than the wild-type strain. This amounted to about a two-fold overall increase in erythromycin production versus the wild type strain.

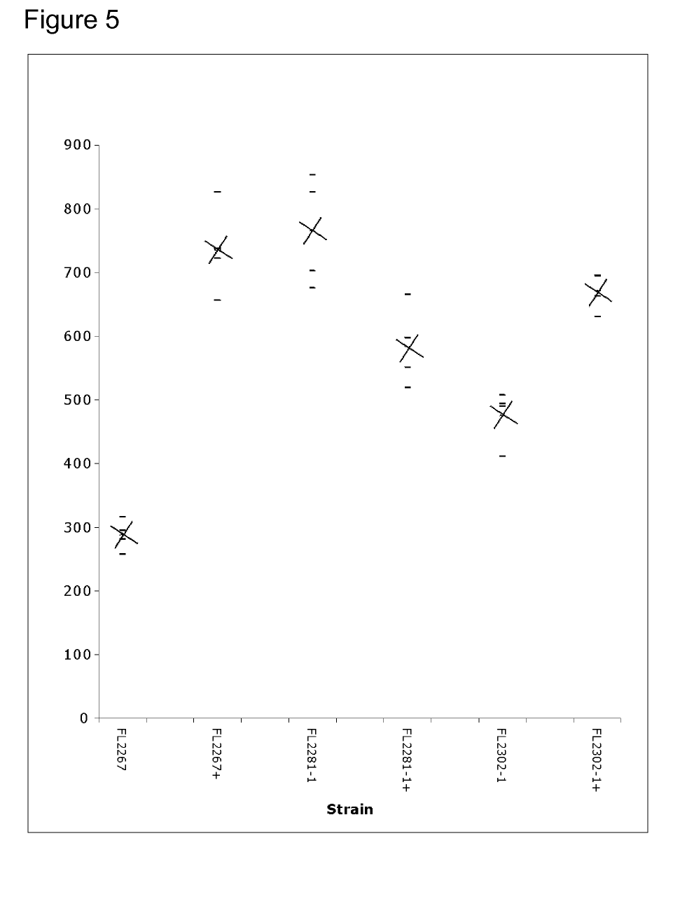

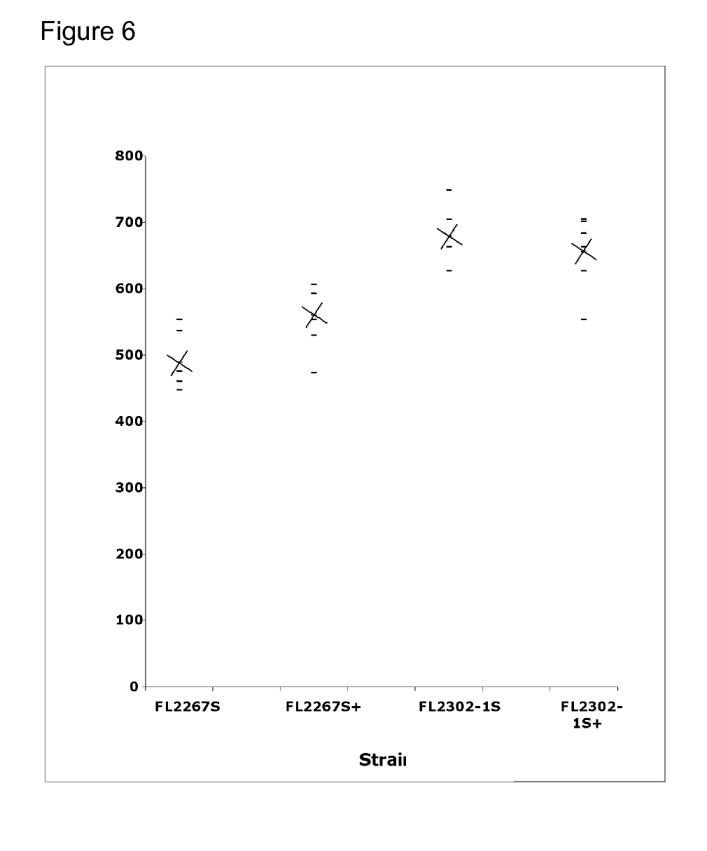

In the fermentations described above, only the mutB mutant FL2281 was tested since the in-frame deletion strain was not available at that time. FL2281 contains an insertion of the aph1 gene (conferring kanamycin resistance) within the mutB gene that would be expected to be polar on the two known and presumably coupled downstream genes (meaB and gntR (SEQ ID NOs:7 and 11). FIG. 5 summarizes the results of experiments testing erythromycin production of FL2281; “X's” in indicate average erythromycin yield for quadruplicate shake flasks for each strain), the trend in the erythromycin yields compared to the wild-type strain was similar to that observed in the previous fermentations, although the overall yields were lower. The in-frame mutant (FL2302) produced about 67% more than the wild type strain in medium 1 but about 50% less than the insertion mutant. When oil was added (medium 2) the in-frame deletion mutant (FL2302) produced nearly as much erythromycin as the wild-type strain and the insertion mutant (FL2281). To test if the in-frame mutant would benefit as much from the addition of 4× starch as the insertion mutant strains were grown in medium 3; the results are shown in FIG. 6; “X's” in indicate average erythromycin yield. In addition, strains were grown in SCM in the presence of both 4× starch and 5% v/v soybean oil (medium 4). The in-frame mutant produced more erythromycin than the parent in both media. The overall increases amounted to 40% in medium 3 and 17% in medium 4.

### Example 4

### Over-Expression of MCM and Erythromycin Production in Wild-Type Industrial Erythromycin-Producing Strain

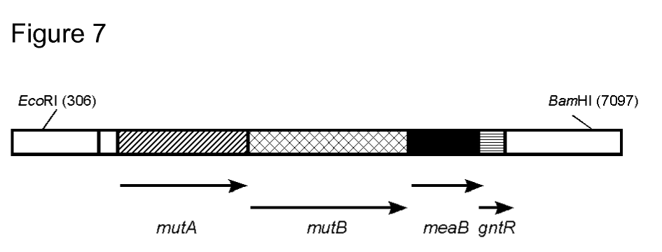

The sequence of the S. erythraea mmCoA region was used as the basis for cloning the entire region including two downstream ORFs, designated meaB and gntR (GenBank Accession No AY117133; SEQ ID NO:8, shown in Table 2). A map of the region is shown in FIG. 7; the diagonal hatch denotes the mutA gene, cross-hatch, mutB gene; solid, meaB; and the horizontal lines, gntR. A 6.791 kb EcoRI+BamHI fragment, also shown in FIG. 7, released from a S. erythraea genomic DNA cosmid library clone was used for sub-cloning. The fragment was ligated into ecoRI+BamHI-digested pFL8 (Reeves et al., 2002). The plasmid containing the cloned mmCoA mutase region was designated pFL2212 (Table 7).

S. erythraea protoplasts were transformed with pFL2212 with selection for thiostrepton antibiotic resistance, indicating introduction of the construct. Wild type strain FL2267 was transformed with varying amounts of pFL2212 DNA (concentration at 0.5 μg/ml) ranging from 5 μg (10 μl) to 10 μg (20 μl). After a 24 hour incubation period at 32° C. protoplasts, were overlaid with thiostrepton at a final concentration of 8 μg/ml. Confluent regeneration and sporulation was only seen in the sectors that were transformed with pFL2212. Thiostrepton-resistant spores were then harvested from the regeneration plates into 20% glycerol and plated onto solid agar (E20A) containing thiostrepton and again selected for strains containing integrated pFL2212. After incubating cultures for ten days, single thiostrepton-resistant colonies were isolated and used for testing in shake flask fermentation. These strains were designated FL2385.

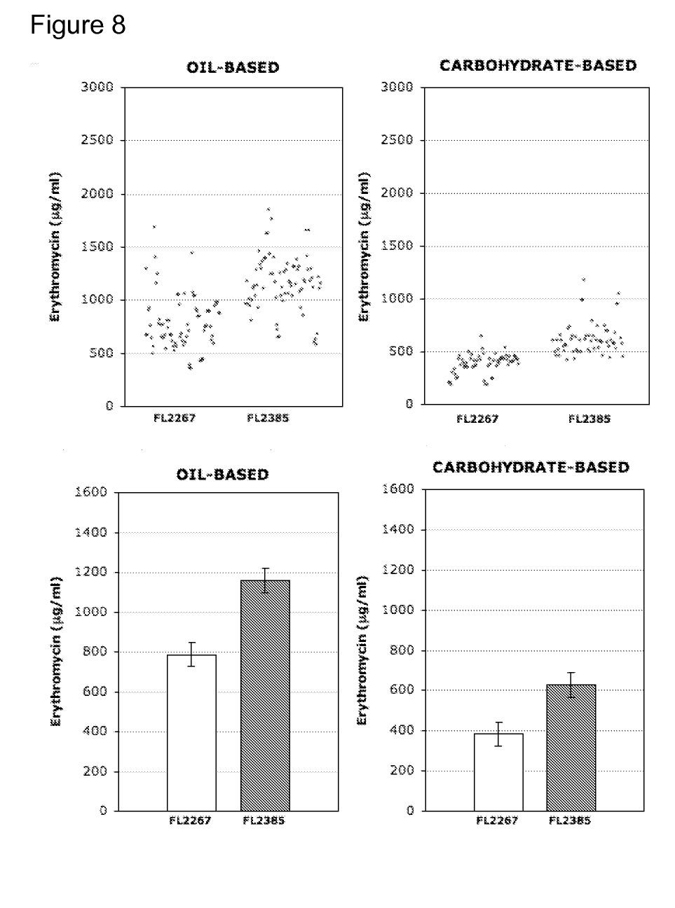

S. erythraea wild type and over-expression strains were grown in IPM+oil and SCM media for 5 days at 32° C. The over-expression strain produced significantly more erythromycin in the IPM media compared to the wild type strain, as shown in FIG. 8; “X's” indicate the average erythromycin production for each condition for triplicate shake flasks. The average production level of the overexpression strain was 1160 μg/ml compared to 786 μg/ml for the parent; representing a 48% increase in production (sample size equal to 74 for both strains). Moreover, the overexpression mutant produced 39% more erythromycin than the parent strain in laboratory medium, SCM (sample size equal to 60 for both strains).

### Example 5

### Knockout of a Regulator of MCM and Erythromycin in Production in an Industrial Erythromycin-Producing Strain (Prophetic)

In addition to generating the over-expression strain, a knockout strain in gntR, encoding a putative transcriptional regulator is generated. The plasmid construct is generated by amplifying two regions: PCR1 and PCR2. PCR1 is 512 bp, covering part of the upstream meaB gene and PCR 2 is 482 bp, spanning all but 6 bp of the gntR ORF as well as some downstream sequences. Restriction sites (e.g., EcoRI and HindIII) are engineered at the 5′ ends of the primers to facilitate cloning into the integrative vector pFL8. A four-component ligation is performed with PCR 1, PCR 2, pFL8 and the kanamycin-resistance gene. E. coli are transformed with the ligation mixture and recombinants are selected on 2×YT media (Sambrook et al., 1989) containing kanamycin and X-gal indicator. Candidate recombinant (white, kanamycin-resistant) isolates are confirmed using restriction digests.

S. erythraea FL2267 protoplasts are then transformed with pFL2123 and selected for kanamycin resistance. Kanamycin is used as the selection agent since gene replacement strains might be obtained in one step as opposed to a two-step process if thiostrepton is used. Transformants are tested on replica plates containing kanamycin or thiostrepton to determine the type of recombination event that occurred.

Transformants are then tested in shake flask fermentations to determine the effect of the mutation on erythromycin production. If gntR is a negative regulator, then its absence results in an increase in erythromycin production; if gntR is a positive regulator, then the opposite effect is observed.

### REFERENCES

WO 90/10448. 1990a. Covalent conjugates of lipid and oligonucleotide.
WO 90/13641. 1990b. Stably transformed eucaryotic cells comprising a foreign transcribable DNA under the control of a pol III promoter.
WO 91/06629. 1991. Oligonucleotide analogs with novel linkages.
Ausubel, F. M., R. Brent, R. E. Kingston, D. D. Moore,J. G. Seidman,J. A. Smith, and K. Struhl. 1987. Current protocols in molecular biology. John Wiley & Sons, New York.
Barbas, I. C., J. Gottesfeld, and P. Wright. U.S. Pat. No. 6,140,466. 2000. Zinc finger protein derivatives and methods therefor.
Bartel, D. P., and J. W. Szostak. 1993. Isolation of new ribozymes from a large pool of random sequences [see comment]. Science. 261:1411-8.
Birch, A., A. Leiser, and J. A. Robinson. 1993. Cloning, sequencing, and expression of the gene encoding methylmalonyl coenzyme A mutase from Streptomyces cinnamonensis. J Bacteriol. 175:3511-9.
Carter, P. 1986. Site directed mutagenesis. Biochem J. 237:1-7.
Cech, T. R., F. L. Murphy, and A. J. Zaug. U.S. Pat. No. 5,116,742. 1992. RNA ribozyme restriction endoribonucleases and methods.
Cech, T. R., A. J. Zaug, and M. D. Been. U.S. Pat. No. 4,987,071. 1991. RNA ribozyme polymerases, dephosphorylases, restriction endoribonucleases and methods.
Cenatiempo, Y. 1986. Prokaryotic gene expression in vitro: transcription translation coupled systems. Biochimie. 68:505-15.
Donadio, S., M. J. Staver, and L. Katz. 1996. Erythromycin production in Saccharopolyspora erythraea does not require a functional propionyl CoA carboxylase. Mol Microbiol. 19:977 84.
Dotzlaf, J. E., L. S. Metzger, and M. A. Foglesong. 1984. Incorporation of amino acid derived carbon into tylactone by Streptomyces fradiae GS14. Antimicrob Agents Chemother. 25:216-20.
Finn, P. J., N. J. Gibson, R. Fallon, A. Hamilton, and T. Brown. 1996. Synthesis and properties of DNA-PNA chimeric oligomers. Nucleic Acids Res. 24:3357 63.
Gautier, C., F. Morvan, B. Rayner, T. Huynh-Dinh, J. Igolen, J. L. Imbach, C. Paoletti, and J. Paoletti. 1987. Alpha-DNA. IV: Alpha-anomeric and beta anomeric tetrathymidylates covalently linked to intercalating oxazolopyridocarbazole. Synthesis, physicochemical properties and poly (rA) binding. Nucleic Acids Res. 15:6625-41.
Gilman, M. Z., J. S. Glenn, V. L. Singer, and M. J. Chamberlin. 1984. Isolation of sigma 28 specific promoters from Bacillus subtilis DNA. Gene. 32:11-20.
Gold, L., D. Pribnow, T. Schneider, S. Shinedbing, B. S. Singer, and G. Stormo. 1981. Translational initiation in prokaryotes. Annu Rev Microbiol 35:365-403.
Gottesman, S. 1984. Bacterial regulation: global regulatory networks. Annu Rev Genet. 18:415 41.
Haseloff,J., and W. L. Gerlach. 1988. Simple RNA enzymes with new and highly specific endoribonuclease activities. Nature. 334:585-91.
Helene, C. 1991. The anti gene strategy: control of gene expression by triplex forming oligonucleotides. Anticancer Drug Des. 6:569-84.
Helene, C., N. T. Thuong, and A. Harel Bellan. 1992. Control of gene expression by triple helix forming oligonucleotides. The antigene strategy. Ann NY Acad Sci. 660:27-36.
Hsieh, Y. J., and P. E. Kolattukudy. 1994. Inhibition of erythromycin synthesis by disruption of malonyl coenzyme A decarboxylase gene eryM in Saccharopolyspora erythraea. J Bacteriol. 176:714-24.
Hunaiti, A. A., and P. E. Kolattukudy. 1984. Source of methylmalonyl coenzyme A for erythromycin synthesis: methylmalonyl-coenzyme A mutase from Streptomyces erythreus. Antimicrob Agents Chemother. 25:173-8.
Hyrup, B., and P. E. Nielsen. 1996. Peptide nucleic acids (PNA): synthesis, properties and potential applications. Bioorg Med Chem. 4:5-23.
Inoue, H., Y. Hayase, A. Imura, S. Iwai, K. Miura, and E. Ohtsuka. 1987a. Synthesis and hybridization studies on two complementary nona(2′-O-methyl)ribonucleotides. Nucleic Acids Res. 15:6131-48.
Inoue, H., Y. Hayase, S. Iwai, and E. Ohtsuka. 1987b. Sequence dependent hydrolysis of RNA using modified oligonucleotide splints and RNase H. FEBS Lett. 215:327-30.
Kellermeyer, R. W., S. H. Allen, R. Stjernholm, and H. G. Wood. 1964. Methylmalonyl Isomerase.Iv. Purification and Properties of the Enzyme from Propionibacteria. J Biol Chem. 239:2562-9.
Lemaitre, M., B. Bayard, and B. Lebleu. 1987. Specific antiviral activity of a poly(L-lysine) conjugated oligodeoxyribonucleotide sequence complementary to vesicular stomatitis virus N protein mRNA initiation site. Proc Natl Acad Sci U S A. 84:648-52.
Letsinger, R. L., G. R. Zhang, D. K. Sun, T. Ikeuchi, and P. S. Sarin. 1989. Cholesteryl conjugated oligonucleotides: synthesis, properties, and activity as inhibitors of replication of human immunodeficiency virus in cell culture. Proc Natl Acad Sci USA. 86:6553-6.
Li, C., G. Florova, K. Akopiants, and K. A. Reynolds. 2004. Crotonyl-coenzyme A reductase provides methylmalonyl-CoA precursors for monensin biosynthesis by Streptomyces cinnamonensis in an oil-based extended fermentation. Microbiology. 150:3463-72.
Maher, L. J. 1992. DNA triple-helix formation: an approach to artificial gene repressors? Bioessays 14:807-15.
Marsh, E. N., N. McKie, N. K. Davis, and P. F. Leadlay. 1989. Cloning and structural characterization of the genes coding for adenosylcobalamin-dependent methylmalonyl-CoA mutase from Propionibacterium shermanii. Biochem J. 260:345-52.
McAlpine, J. B., J. S. Tuan, D. P. Brown, K. D. Grebner, D. N. Whittern, A. Buko, and L. Katz. 1987. New antibiotics from genetically engineered actinomycetes. I. 2-Norerythromycins, isolation and structural determinations. J Antibiot (Tokyo). 40:1115-22.
Omura, S., A. Taki, K. Matsuda, and Y. Tanaka. 1984. Ammonium ions suppress the amino acid metabolism involved in the biosynthesis of protylonolide in a mutant of Streptomyces fradiae. J Antibiot (Tokyo). 37:1362-9.
Omura, S., K. Tsuzuki, Y. Tanaka, H. Sakakibara, M. Aizawa, and G. Lukacs. 1983. Valine as a precursor of n-butyrate unit in the biosynthesis of macrolide aglycone. J Antibiot (Tokyo). 36:614-6.
Perry O'Keefe, H., X. W. Yao, J. M. Coull, M. Fuchs, and M. Egholm. 1996. Peptide nucleic acid pre gel hybridization: an alternative to southern hybridization. Proc Natl Acad Sci U S A. 93:14670-5.
Petersen, K. H., D. K. Jensen, M. Egholm, O. Buchardt, and P. E. Nielsen. 1976. A PNA-DNA inker synthesis of N-((4,4′-dimethoxytrityloxy)ehtyl)N(thymin-1-ylacetyl)glycine. Biorganic and Medicianl Chemistry Letters. 5:1119-1124.
Reeves, A. R., I. A. Brikun, W. H. Cernota, B. I. Leach, M. C. Gonzalez, and J. M. Weber. 2006. Effects of methylmalonyl CoA mutase gene knockouts on erythromycin production in carbohydrate-based and oil-based fermentations of Saccharopolyspora erythraea. J Ind Microbiol Biotechnol. 33:600-9.
Reeves, A. R., W. H. Cernota, I. A. Brikun, R. K. Wesley, and J. M. Weber. 2004. Engineering precursor flow for increased erythromycin production in Aeromicrobium erythreum. Metab Eng 6:300-12.
Reeves, A. R., G. Weber, W. H. Cernota, and J. M. Weber. 2002. Analysis of an 8.1-kb DNA fragment contiguous with the erythromycin gene cluster of Saccharopolyspora erythraea in the eryCI-flanking region. Antimicrob Agents Chemother. 46:3892-9.
Sambrook,J., E. F. Fritsch, and T. Maniatis. 1989. Molecular cloning: a laboratory manual. Cold Spring Harbor Laboratory, Cold Spring Harbor, N.Y.
Stein, C. A., and J. S. Cohen. 1988. Oligodeoxynucleotides as inhibitors of gene expression: a review. Cancer Res. 48:2659-68.
Tang, L., Y. X. Zhang, and C. R. Hutchinson. 1994. Amino acid catabolism and antibiotic synthesis: valine is a source of precursors for macrolide biosynthesis in Streptomyces ambofaciens and Streptomyces fradiae. J Bacteriol. 176:6107-19.
Uitenbroek, D. 2005. SISA Online Statistical Analysis. Uitenbroek, D, Hilversum, The Netherlands.
Ulmanen, I., K. Lundstrom, P. Lehtovaara, M. Sarvas, M. Ruohonen, and I. Palva. 1985. Transcription and translation of foreign genes in Bacillus subtilis by the aid of a secretion vector. J Bacteriol 162:176-82.
van der Krol, A. R., J. N. Mol, and A. R. Stuije. 1988a. Modulation of eukaryotic gene expression by complementary RNA or DNA sequences. Biotechniques. 6:958-76.
van der Krol, A. R., J. N. Mol, and A. R. Stuije. 1988b. Modulation of eukaryotic gene expression by complementary RNA or DNA sequences. Biotechniques. 6:958-76.
Vlasie, M. D., and R. Banerjee. 2003. Tyrosine 89 accelerates Co-carbon bond homolysis in methylmalonyl-CoA mutase. J Am Chem Soc. 125:5431-5.
Ward, J. M., G. R. Janssen, T. Kieser, M. J. Bibb, M. J. Buttner, and M. J. Bibb. 1986. Construction and characterisation of a series of multi-copy promoter probe plasmid vectors for Streptomyces using the aminoglycoside phosphotransferase gene from Tn5 as indicator. Mol Gen Genet. 203:468-78.
Weber, J. M., J. O. Leung, G. T. Maine, R. H. Potenz, T. J. Paulus, and J. P. DeWitt. 1990. Organization of a cluster of erythromycin genes in Saccharopolyspora erythraea. J Bacteriol. 172:2372-83.
Weber, J. M., and R. Losick. 1988. The use of a chromosome integration vector to map erythromycin resistance and production genes in Saccharopolyspora erythraea (Streptomyces erythraeus). Gene. 68:173-80.
Wells, J. A., M. Vasser, and D. B. Powers. 1985. Cassette mutagenesis: an efficient method for generation of multiple mutations at defined sites. Gene. 34:315-23.
Zhang, W., L. Yang, W. Jiang, G. Zhao, Y. Yang, and J. Chiao. 1999. Molecular analysis and heterologous expression of the gene encoding methylmalonyl-coenzyme A mutase from rifamycin SV-producing strain Amycolatopsis mediterranei U32. Appl Biochem Biotechnol. 82:209-25.
Zoller, M. J., and M. Smith. 1987. Oligonucleotide directed mutagenesis: a simple method using two oligonucleotide primers and a single-stranded DNA template. Methods Enzymol. 154:329-50.
Zon, G. 1988. Oligonucleotide analogues as potential chemotherapeutic agents. Pharm Res. 5:539-49.

We claim:

1. A method of increasing the production of a secondary metabolite derived at least in part from methylmalonyl-CoA in a cell comprising increasing a metabolite pool of methylmalonyl-CoA in the cell, wherein the production of the secondary metabolite increases.

2. The method of claim 1, wherein increasing the metabolite pool of methylmalonyl-CoA in the cell comprises culturing the cell in an oil-based medium.

3. The method of claim 1, wherein filling the metabolite pool comprises modifying the cell to diminish the activity of an enzyme that depletes the size of the methylamalonyl-CoA pool, wherein the enzyme is other than one that leads to production of the secondary metabolite.

4. The method of claim 1, wherein filling the metabolite pool comprises modifying the cell to increase the activity or concentration of an enzyme that increases the size of the methylmalalonyl-CoA pool.

5. The method of claim 3, wherein modifying the cell comprises genetically altering a gene encoding at least part of the enzyme.

6. The method of claim 5, wherein genetically altering the gene comprises preventing its expression.

7. The method of claim 5, wherein the altered gene comprises at least one selected from the group consisting of mutB, mutA, meaB, and gntR.

8. The method of claim 7, wherein the secondary metabolite comprises erythromycin.

9. The method of claim 1, wherein the cell is one selected from the group consisting of Streptomyces fradiae, Streptomyces avermitilis, Streptomyces cinnamonensis, Streptomyces antibioticus, Streptomyces venezuelae, Streptomyces violaceoniger, Streptomyces hygroscopicus, Streptomyces spp. FR-008, Saccharopolyspora erythraea and Streptomyces griseus.

10. A method of increasing the cellular production of a secondary metabolite derived at least in part from methylmalonyl-CoA comprising increasing the activity of methylmalonyl-CoA mutase in a cell.

11. The method of claim 10, wherein increasing the activity of methymalonyl-CoA mutase comprises increasing the expression of the mutase.

12. The method of claim 11, wherein increasing the activity of the mutase comprises over-expressing the mutase.

13. The method of claim 12, wherein increasing the expression of the mutase comprises one selected from the group consisting of introducing an endogenous or heterologous mutase, decreasing the expression of a negative regulator, increasing the expression of a positive regulator, culturing the cell in a media that increases the expression of the mutase, or a combination thereof.

14. The method of claim 13, wherein decreasing the expression of the negative regulator comprises inhibiting the transcription or translation of the negative regulator.

15. The method of claim 13, wherein decreasing the expression of the negative regulator comprises expressing an anti-sense polynucleotide to the negative regulator, or expressing a dominant negative construct.

16. The method of claim 13, wherein increasing expression of the positive regulator comprises increasing the transcription or translation of the positive regulator.

17. The method of claim 13, wherein increasing the expression of the positive regulator comprises over-expressing the positive regulator.

18. The method of claim 1, wherein the cell is S. erythraea and increasing the activity of the mutase is accomplished by culturing the cells in a media that increases mutase activity when compared to culturing the cells in soluble complete medium.

19. The method of claim 1, wherein the secondary metabolite is an antibiotic.

20. The method of claim 19, wherein the antibiotic is a polyketide antibiotic.

21. The method of claim 20, wherein the polyketide antibiotic is a macrolide polyketide antibotic.

22. The method of claim 21, wherein the macrolide polyketide antibiotic is one selected from the group consisting of erythromycin, tylosin, niddamycin, spiramycin, oleandomycin, methymycin, neomethymycin, narbomycin, pikromycin and lankamycin.

23. The method of claim 1, wherein the cell is a prokaryotic cell.

24. The method of claim 23, wherein the prokaryotic cell is a bacterial cell.

25. The method of claim 24, wherein the bacterial cell is Saccharopolyspora, Aeromicrobium or Streptomyces.

26. The method of claim 25, wherein the bacterial cell is Saccharopolyspora erythraea or Aeromicrobium erythreum.

27. The method of claim 26, wherein the bacterial cell is Streptomyces fradiae, Streptomyces avermitilis, Streptomyces cinnamonensis, Streptomyces antibioticus, Streptomyces venezuelae, Streptomyces violaceoniger, Streptomyces hygroscopicus, Streptomyces spp. FR-008, or Streptomyces griseus.

28. The method of claim 1, wherein the cell is a eukaryotic cell.

29. The method of claim 28, wherein the eukaryotic cell is a plant cell.

30. The method of claim 28, wherein the eukaryotic cell is an animal cell.

31. The method of claim 30, wherein the animal cell is a mammalian cell.

32. A method of increasing the production of a secondary metabolite derived at least in part from methylmalonyl-CoA in a Saccharopolyspora erythea cell, comprising increasing the activity of methylmalonyl-CoA mutase in the cell

33. The method of claim 32, wherein increasing the activity of the mutase comprises over-expressing the mutase, and culturing the cells in media other than SCM medium.

34. The method of claim 32, wherein increasing the activity of the mutase comprises inhibiting the activity or expression of a negative regulatory gene, and culturing the cells in media other than SCM medium.

35. The method of claim 32, wherein the secondary metabolite comprises an antibiotic.

36. The method of claim 35, wherein the antibiotic is a polyketide antibiotic.

37. The method of claim 36, wherein the polyketide antibiotic is a macrolide polyketide antibiotic.

38. The method of claim 37, wherein the macrolide polyketide antibiotic is erythromycin.

39. A cell modified to increase the activity of methylmalonyl-CoA.

40. The cell of claim 39, wherein the increase in activity comprising increasing the expression of methylmalonyl-CoA.

41. The cell of claim 40, wherein increasing the expression of the mutase comprises introducing an additional copy of an endogenous or heterologous mutase, decreasing the expression of a negative regulator, increasing the expression of a positive regulator, culturing the cell in a media that increases the expression of the mutase, or a combination thereof.

42. The cell of claim 41, wherein decreasing the expression of the negative regulator comprises inhibiting the transcription or translation of the negative regulator.

43. The cell of claim 41, wherein decreasing the expression of the negative regulator comprises expressing an anti-sense polynucleotide to the negative regulator, or expressing a dominant negative construct.

44. The cell of claim 41, wherein increasing expression of the positive regulator comprises increasing the transcription or translation of the positive regulator.

45. The cell of claim 41, wherein increasing the expression of the positive regulator comprises over-expressing the positive regulator.

46. The cell of claim 39, wherein the cell is S. erythraea and increasing the activity of the mutase is accomplished by culturing the cells in a media that increases mutase activity when compared to culturing the cells in soluble complete medium.

47. The cell of claim 39, wherein the secondary metabolite is an antibiotic.

48. The cell of claim 47, wherein the antibiotic is a polyketide antibiotic.

49. The cell of claim 48, wherein the polyketide antibiotic is a macrolide polyketide antibiotic.

50. The cell of claim 49, wherein the macrolide polyketide antibiotic is one selected from the group consisting of erythromycin, tylosin, niddamycin, spiramycin, oleandomycin, methymycin, neomethymycin, narbomycin, pikromycin and lankamycin.

51. The cell of claim 39, wherein the cell is a prokaryotic cell.

52. The cell of claim 51, wherein the cell is a bacterial cell.

53. The cell of claim 52, wherein the cell is Saccharopolspora, Aeromicrobium or Streptomyces.

54. The cell of claim 53wherein the bacterial cell is a Saccharopolyspora erythraea or an Aeromicrobium erythreum.

55. The cell of claim 54, wherein the bacterial cell is Streptomyces fradiae, Streptomyces avermitilis, Streptomyces cinnamonensis, Streptomyces antibioticus, Streptomyces venezuelae, Streptomyces violaceoniger, Streptomyces hygroscopicus, Streptomyces spp. FR-008, or Streptomyces griseus.
